(19) United States
(12) Patent Application Publication
(10) Pub. No.: US 2010/0268287 A1
(43) Pub. Date: Oct. 21, 2010

(54) METHODS AND DEVICES FOR INCREASING LEARNING AND EFFECTS OF TRAINING IN HEALTHY INDIVIDUALS AND PATIENTS AFTER BRAIN LESIONS USING DC STIMULATION AND APPARATUSES AND SYSTEMS RELATED THERETO

(75) Inventor: Pablo A. Celnik, Baltimore, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/724,389

(22) Filed: Mar. 15, 2010

Related U.S. Application Data
(60) Provisional application No. 61/159,882, filed on Mar. 13, 2009.

(51) Int. Cl. A61N 1/00; A61N 1/05

(52) U.S. Cl. 607/2; 607/115

(57) ABSTRACT

Featured is a method and device for increasing learning and effects of training. Such methods include locating a pair of electrodes on a head of a person in relation to a specific area of the brain, applying a desired DC current to the electrodes at a level sufficient to stimulate the brain tissue; and controlling the DC current application so the current is applied to the specific brain area at least one of before, during or after such a learning or training event. In this way, application of the DC current to the brain area improves a subject's ability to acquire one of motor skills or knowledge of the learning or training event or the ability to retain the motor skills or knowledge of the learning or training event. The person to which the electrodes are attached can be healthy or a patient after brain lesions.

This application claims the benefit of U.S. Provisional Application Ser. No. 61/159,882 filed Mar. 13, 2009, the teachings of which are incorporated herein by reference.

### STATEMENT REGARDING FEDERALLY-SPONSORED RESEARCH

The present invention was supported under R01HD053793 from NICHD and supported under 5K12HD001097 from RMSTP. The U.S. Government may have certain rights to the present invention.

### FIELD OF INVENTION

The present invention relates to methods and devices for increasing learning and effects of training in healthy individuals and patients after brain lesions and more particularly to such methods and devices that utilize DC stimulation so as to increase such learning and effects of training.

### BACKGROUND OF THE INVENTION

Accurate motor performance is essential to almost everything that is done, from typing, to driving, to playing sports. Having a motor skill implies a level of performance in a given task that is only achievable through practice [Schmidt R A, Lee TD (2005) Motor Control and Learning: A Behavioral Emphasis (Human Kinetics)]. Evidence indicates that motor skill learning can continue over a prolonged time period [Korman M, Raz N, Flash T, Karni A (2003) Multiple shifts in the representation of a motor sequence during the acquisition of skilled performance Proc Natl Acad Sci USA 100, 12492-12497; Luft A R, Buitrago M M (2005) Stages of motor skill learning Mol Neurobiol 32, 205-216; Shea C H, Kohl R M (1990) Specificity and variability of practice Res. Q. Exerc. Sport 61, 169-177; Savion-Lemieux T, Penhune VB (2005) The effects of practice and delay on motor skill learning and retention Exp Brain Res 161, 423-431].

Within-session performance improvements (online effects) occur in the minutes or hours of a single training session and continue over days and weeks of repeated training sessions until performance nears asymptotic levels. Changes in performance can also occur between training sessions (offline effects), i.e., performance at the beginning of session n+1 is different than performance at the end of session n [Robertson E M, Pascual-Leone A, Miall R C (2004) Current concepts in procedural consolidation Nat Rev Neurosci 5, 576-582; Adams J A (1952) Warm-up decrement in performance on the pursuit-rotor Am J Psychol 65, 404-414]. The use of the term “offline learning” is generally avoided herein because it has been used to refer to both a physiological process (consolidation) and a particular measurement result (a positive offline effect). Offline effects could also be negative, presumably due to forgetting processes. Finally, skills can be retained to varying degrees over weeks to months after the completion of training (long-term retention).

Noninvasive brain stimulation methods have been used frequently to modulate cortical excitability [Pascual-Leone A, Valls-Solé J, Wassermann E M, Hallett M (1994) Responses to rapid-rate transcranial magnetic stimulation of the human motor cortex Brain 117 (Pt 4), 847-858; Rosenkranz K, Nitsche M A, Tergau F, Paulus W (2000) Diminution of training-induced transient motor cortex plasticity by weak transcranial direct current stimulation in the human Neurosci Lett 296, 61-63; Nitsche M A, Paulus W (2000) Excitability changes induced in the human motor cortex by weak transcranial direct current stimulation J. Physiol 527 Pt 3, 633-639]. These methods, and in particular transcranial magnetic stimulation, have been shown to perturb initial motor learning and consolidation [Robertson E M, Press D Z, Pascual-Leone A (2005) Off-line learning and the primary motor cortex J Neurosci 25, 6372-6378; Muellbacher W, et al. (2002) Early consolidation in human primary motor cortex Nature 415, 640-644; Richardson A G, et al. (2006) Disruption of primary motor cortex before learning impairs memory of movement dynamics J Neurosci 26, 12466-12470].

Anodal transcranial direct current stimulation (tDCS) delivered over the primary motor cortex (M1) increases motor cortical excitability without direct neuronal depolarization at the low intensities used in humans, whereas cathodal tDCS decreases cortical excitability [Nitsche M A, Paulus W (2000) Excitability changes induced in the human motor cortex by weak transcranial direct current stimulation J. Physiol 527 Pt 3, 633-639]. A single application of anodal tDCS over M1 has been shown to induce transient performance improvements in various motor tasks [Antal A, et al. (2004) Facilitation of visuo-motor learning by transcranial direct current stimulation of the motor and extrastriate visual areas in humans Eur J Neurosci 19, 2888-2892; Nitsche M A, et al. (2003) Facilitation of implicit motor learning by weak transcranial direct current stimulation of the primary motor cortex in the human J Cogn Neurosci 15, 619-626; Boggio P S, et al. (2006) Enhancement of non-dominant hand motor function by anodal transcranial direct current stimulation Neurosci Lett 404, 232-236; Vines B W, Nair D G, Schlaug G (2006) Contralateral and ipsilateral motor effects after transcranial direct current stimulation Neuroreport 17, 671-674]. Because these studies only examined the effects of tDCS within a single session, the relative impact of anodal tDCS on online effects, offline effects, and long-term retention is not known.

The cerebellum also plays an important role in the planning, initiation, stability, organization and long-term memory of movements [Chen et al., 2006; Diedrichsen et al., 2005; Imamizu et al., 2000; Miall et al., 2007; Morton and Bastian, ]. Therefore, developing strategies to modulate cerebellar excitability is of significant interest to further the understanding of its function and as potential rehabilitation strategy for patients with cerebellar and cerebral diseases.

Previous studies aiming to modulate cerebellar excitability have applied slow repetitive transcranial magnetic stimulation (rTMS) over the cerebellum and determined the neurophysiological effects indirectly on M1 [Fierro et al., 2007; Koch et al., 2008; Langguth et al., 2008; Oliveri et al., 2005]. However, the findings have been inconsistent, where some described an increase in intracortical M1 excitability [Oliveri et al., 2005; Koch et al., 2008] and others the opposite [Fierro et al., 2007; Langguth et al., 2008]. Other investigations have reported the behavioral effects of cerebellar rTMS such as increased variability of finger tapping [Theoret et al., 2001] and reduced essential tremor [Gironell et al., 2002].

One of the main outputs of the cerebellum is the dentate-thalamo-cortical pathway. The Purkinje cells, the output neurones of the cerebellar cortex, have inhibitory connections with the dentate cerebellar nucleus (DCN), which in turn has a disynaptic excitatory connection through the thalamus to M1 [Kelly and Strick, 2003; Middleton and Strick, 2000; Reis et al, 2008]. Thus, Purkinje cell activity exerts an inhibitory tone over M1, referred as cerebellum-brain inhibition (CBI). Evidence for the presence of CBI comes from studies evaluating motor cortical excitability in healthy and patients with cerebellar stroke or degeneration. The latter have consistently shown enhancement of corticomotor inhibition and reduction in motor facilitation secondary to lesions of the DCN [Battaglia et al., 2006; Liepert et al., 2004]. In healthy humans, the dentate-thalamo-cortical pathway has been assessed non-invasively through electrical and magnetic stimulation of the cerebellum [Daskalakis et al., 2004; Pinto and Chen, 2001; Ugawa et al., 1991, 1995]. Therefore, it would be possible to directly determine the effect of cerebellar stimulation by evaluating changes in CBI.

It thus would be desirable to provide new devices and methods for increasing learning and the effects of training, that utilize DC stimulation of the brain tissue. It would be particularly desirable to provide such a device and method that would increase learning and the effects of training in healthy individuals and/or patients after brain lesions. It also would be particularly desirable to provide such devices and methods that would one of improving one's ability to acquire knowledge or skill in such learning and training as well as increasing one's ability to retain acquired knowledge or skill in such learning and training. Such devices preferably would be simple in construction and less costly than prior art devices and such methods would not require more highly skilled users to utilize the device as compared to the skill of those using conventional methods.

### SUMMARY OF THE INVENTION

The present invention features devices and methods for increasing learning and the effects of training utilizing DC stimulation of the brain tissue. Such featured methods and devices also would increase learning and the effects of training in healthy individuals and/or patients after brain lesions. More particularly, such methods and devices advantageously would one or both of improve one's ability to acquire knowledge or skill in such learning and training and/or increase one's ability to retain acquired knowledge or skill in such learning and training. Subjects, persons or patients using such devices and methods can achieve larger results given the same amount of practice when not using such devices or methods. Such would be the case for skill physical activities like typing, playing instruments, sports, etc, and other cognitive tasks, like learning facts, strings or words etc. More particularly, application of such DC stimulation should benefit people practicing or learning sports, how to play musical instruments, dance, hand skills or occupations that require fine motor skills (i.e., typist, dentists, seamstress, assembly line workers, painters, hand artists like sculpture, video game playing, handling or interfacing with robots, machinist, crane operators, mechanics, drivers, pilots, etc). Similarly, patients who suffered neurological lesions and need to perform rehabilitation exercises will benefit further when the training is done under the influence of DC stimulation whether the stimulation is applied immediately before, during or after the completion of the exercises. In addition, DC stimulation also should benefit other activities that are not exclusively motoric in nature, such as language and speech learning, visual coordination and discrimination, activities where perception and sensitivity demands are high, swallowing, and audition.

In more particular embodiments, such methods include delivering direct current stimulation to specific areas of the brain and other parts of the central nervous system one of during, before or after the performance of training. In yet further embodiments, such delivering includes (a) delivering direct current stimulation before and during the performance of training (b) delivering direct current stimulation during and after the performance of training; or (c) delivering direct current stimulation during, before and after the performance of training.

In yet further embodiments, such methods include delivering such DC current stimulation using a DC stimulator that is connected to at least 2 electrodes, where one electrode delivers anodal stimulation and the other cathodal stimulation. The selection of the type of stimulation (anodal or cathodal), duration and intensity is specified depending on the location of stimulation site and activity being practiced. In yet further embodiments, the DC simulator is selectively coupled to a plurality of pairs of electrodes, each pair of electrodes be arranged about the exterior of the head so as to stimulate a plurality of areas of the brain.

In yet further embodiments, each pair of electrodes is located in or otherwise secured to headgear (e.g., cap, hat, head band, bandana) so that the headgear provides a mechanism such that the electrodes will be in preselected localization to the head depending on the activity being trained. Such headgear also is configurable so it and the electrodes can be adjusted to head size or manufactured in different sizes ensuring predetermined electrode position for different head sizes.

In yet further embodiments, there is featured a stimulation system including a DC stimulation device, cables and electrodes. Preferably, the system as well as components thereof is portable and can be attached to the body of the person, subject or patient by any of a number of devices known to those skilled in the art including, but not limited to a bracelet, belt, waist bags, or placed in clothing pockets or backpacks. In further embodiments, portions of such a system are connected to the headgear, and the headgear will be exchangeable. In this way, the same system is configurable so it can be attached to different headgears.

In more particular embodiments, such stimulation devices and/or systems are configurable so as to be small, lightweight and portable and also so that a transcranial direct current stimulation (tDCS) can be applied over specific areas of the brain during training of normal activities or during rehabilitation exercises so as to thereby enhance the magnitude of gains achieved and thus result in stronger effects of practice increasing learning.

In yet further embodiments, such devices and systems include a DC power supply such as a power supply composed of one or more batteries (e.g., a conventional nine volt battery). Such devices and systems also include a controller so as to control and maintain a relatively steady application of the DC stimulation including the DC current and voltage being applied to stimulate the brain areas. In yet further embodiments, the DC current is ramped up slowly over the course of a predetermined period of time (e.g., several seconds) to the relatively steady value, when the stimulation period starts.

As indicated above, such a system or device can include 2 electrodes or one or more pairs of electrodes. The controller is configurable so that one electrode delivers anodal stimulation to the brain tissue and the other cathodal stimulation. The selection of the type of stimulation (anodal or cathodal), duration and intensity will be specified depending on the location of stimulation site and activity being practiced. Thus, the controller is configurable so as to control such selection, duration and intensity.

Other aspects and embodiments of the invention are discussed below.

### DEFINITIONS

The instant invention is most clearly understood with reference to the following definitions:

The term M1 shall be understood to mean the primary motor cortex.

The term tDCS shall be understood to mean transcranial direct current stimulation.

The term DCN shall be understood to mean dentate cerebellar nucleus.

The term CBI shall be understood to mean as cerebellum-brain inhibition, Purkinje cell activity that exerts an inhibitory tone over M1.

As used in the specification and claims, the singular form “a”, “an” and “the” include plural references unless the context clearly dictates otherwise.

As used herein, the term “comprising” or “including” is intended to mean that the compositions, methods, devices, apparatuses and systems include the recited elements, but do not exclude other elements. “Consisting essentially of”, when used to define compositions, devices, apparatuses, systems, and methods, shall mean excluding other elements of any essential significance to the combination. Embodiments defined by each of these transition terms are within the scope of this invention.

The term patient shall be understood to include mammalians including human beings as well as other members of the animal kingdom.

USP shall be understood to mean U.S. Pat. No., namely a U.S. patent granted by the U.S. Patent and Trademark Office.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is a schematic block diagram of an exemplary stimulation device according to the present invention.

FIG. 2 is a high level flow chart or diagram of a method for increasing learning and effects of training in healthy individuals and patients after brain lesions using dc stimulation.

FIG. 3 is an illustrative view of the Sequential Visual Isometric Pinch Task. To control an on-screen cursor movement subjects pinched a force transducer with thumb and index finger. The aim was to navigate the cursor quickly and accurately between a HOME position and 5 gates by alternating the pinch force exerted onto the transducer. The practiced sequence was Home-1-Home-2-Home-3-Home-4-Home-5. Movement time (from movement onset to stopping at gate 5) and error rate (proportion of trials with at least one under- or overshooting movement) were used to determine a skill measure.

FIGS. 4 A, B are various views of a target region for cortical stimulation (FIG. 4A) and of a study design (FIG. 4B). In the target region for cortical stimulation, left M1 was determined by TMS targeting the optimal scalp position to elicit MEPs (inset) of the right first dorsal interosseus muscle. Neuronavigation revealed the precentral gyms as the cortical target for tDCS. The anode/cathode was placed according to this landmark and the second electrode was placed on the right supraorbital area. In the study design, subjects participated in 5 training and 5 follow up sessions. During training, 20 min of sham or anodal tDCS was applied to M1 in a double blind fashion while subjects practiced the SVIPT. In the follow ups, subjects performed 40 trials. Fam.: Familiarization with the experimental setting.

FIG. 5 is a graphical view of the speed-accuracy-tradeoff function. Speed-accuracy tradeoff function data (black diamonds: pre-training initial dataset; black triangles: post-training initial dataset; unfilled squares: post-training validation dataset) and the corresponding nonlinear least squares fits of Eq. [1] (gray lines: initial dataset fits; black line: validation dataset fit). Data were obtained from 12 naïve subjects for the initial dataset and 6 subjects per post-training dataset. The SAF is derived from the Movement time (abscissa) and the error rate per block (ordinate).

FIG. 6 is a graphical view of the learning curve. Learning curve of the sham (white diamonds) and anodal (grey squares) tDCS group for the 30 training blocks over 5 days. Each block depicts the group mean of the averaged number of trials (40 in block 1 and 6, 30 in blocks 2-5). The dotted lines represent breaks between consecutive days. Both groups started with a comparable skill at the beginning of day 1, but by day 5 the anodal tDCS group had a much higher skill than the sham tDCS group.

FIG. 7 is a graphical view of online and offline effects. Online (within day) and offline (between day) effects and total learning (online+offline) in the sham tDCS (white bars) and anodal tDCS (grey bars) groups are shown. Note that the significantly greater total learning in the anodal tDCS group (last grey bar) was predominantly driven by the significantly different offline effect compared to sham tDCS, in the absence of differences in online effects. Data show mean (Bars)±SEM. *p<0.05, **p<0.01.

FIG. 8 is a graphical view of Retention of skill. Skill at D5 and at follow up sessions on D8, D15±1, D29±2, D57±2 and D85±2 is shown. Skill in the anodal tDCS group (grey squares) remained superior to the sham tDCS group (white diamonds) at all times, including D85. Small inset: Retention, the time weighed slope measure calculated within single subjects over the follow up period, did not differ between the sham (white bar) and anodal (grey bar) tDCS groups. Data show mean±SEM. *p<0.01.

FIGS. 9A, B are schematic representation of the main experimental setup for Experiment 1 (FIG. 9A) and Experiment 2 (FIG. 9B). For Experiment 1, three groups (n=10; sham, cb, m1) experienced a similar protocol involving six blocks. Pre1, Pre2, Post1 and Post2 were all under veridical conditions. During Adapt1 and Adapt2 participants were exposed to a 30-degree counter clockwise (CCW) screen-cursor transformation. Anodal (cb, m1) or sham tDCS (sham) was applied to either the ipsilateral cerebellar cortex (cb) or contralateral motor cortex (m1) during Pre2 and Adapt1 (approximately 15 minutes; shaded area). There was a one minute rest period between each block. The numbers under each block represent the amount of trials, while the numbers in brackets indicate the approximate length of each block. For Experiment 2, three groups (n=10; sham, cb, m1) experienced a similar protocol involving six blocks. Pre1, Pre2 were under veridical conditions. During Adapt1 participants were exposed to a 30-degree CCW screen-cursor transformation and anodal or sham tDCS was applied during Pre2 and Adapt1. Post 1, 2 and 3 all involved trials with no visual feedback.

FIGS. 10A-C are various illustrative views of single subject data for Experiment 1. In FIG. 10A, Pre2: epoch 12; a sample participant from the sham (black), cb (cerebellar anodal tDCS: red) and m1 (M1 anodal tDCS: blue) groups in experiment one. Under veridical conditions, all groups made similar accurate movement trajectories towards each target. In FIG. 10B, Adapt1: epoch, when initially exposed to the novel 30-degree CCW screen-cursor transformation participants show comparable error in their trajectories. In FIG. 10C, Adapt1: epoch 8, in comparison to the sham and m1 participants, the cb participant is able to display a reduced amount of error in their movement trajectories.

FIGS. 11A, B are various views of Group data for Experiment 1. In FIG. 11A, Group data: Experiment one, Epoch error; end-point error (degrees) during baseline (Pre1,2), adaptation (1,2) and deadaptation (Post1,2) for the sham (black), cb (red) and m1 (blue) groups (mean±SEM of 8 trial epochs). Positive values indicate counter-clockwise deviation and the shaded area represents blocks in which tDCS was applied (Pre2, Adapt1). In FIG. 11B, Mean error, values depict the mean (±SEM) end-point error (degrees) for the groups (sham (black), cb (red), m1 (blue)) in each block. This was determined for each participant by averaging over consecutive epochs. For Pre1, Pre2, Post1 and Post2 epochs 2-6 were averaged, whereas for Adapt 1 and Adapt2 epochs 2-11 were averaged. For each block, separate one-way ANOVAs compared these values across groups. *=p<0.009.

FIGS. 12A-D are various views of single subject data for Experiment two. In FIG. 12A, Single subject data: Experiment two, Pre2: epoch 12, a sample participant from the sham (black), cb (red) and m1 (blue) groups in Experiment two. Under veridical conditions, all groups made similar accurate movement trajectories towards each target. In FIG. 12B, Adapt1: epoch 8, similarly to Experiment one, the cb participant exhibits a reduced amount of error in their movement trajectories by epoch eight, which is not observed in both the sham and m1 participants. In FIG. 12C, Post1: epoch 1, initially in the trials with no vision, all participants show a similar amount of error. In FIG. 12D, Post3: epoch 8, by the end of the no-vision blocks, the m1 participant displays a larger amount of movement error relative to the sham and cb participants.

FIGS. 13 A, B are various views of Group data for experiment two. In FIG. 13A, Group data for experiment two, Epoch error; there is shown end-point error (degrees) during baseline (Pre1,2), adaptation (1) and deadaptation with no vision (Post1,2,3) for the sham (black), cb (red) and m1 (blue) groups (mean±SEM of 8 trial epochs). Positive values indicate counter-clockwise deviation and the shaded area represents blocks in which tDCS was applied (Pre2, Adapt1). In FIG. 13B, Mean error, values depict the mean (±SEM) end-point error (degrees) for the groups (sham (black), cb (red), m1 (blue)) in each block. This was determined for each participant by averaging over consecutive epochs. For Pre1 and Pre2 epochs 2-6 were averaged, whereas for Adapt 1, Post1, Post2 and Post3 epochs 2-11 were averaged. For each block, separate one-way ANOVAs compared these values across groups. *=p<0.05.

FIG. 14 is a tabulation of Psychological measures. Values (mean±SEM) depict subject's choice in a visual analog scale where 1 represents poorest attention, maximal fatigue and pain, and 7 maximal attention, least fatigue and pain. F and p values originate from separate ANOVAs for each measure comparing the sham, cb and m1 groups.

FIG. 15 is a tabulation of Kinematic parameters: Experiment one. Movement duration (msecs), reaction time (msecs) and maximum velocity (cm/s) were assessed for the sham, cb and m1 groups in each block of experiment one. Values depict the mean (±SEM) which was determined for each participant by averaging over consecutive epochs. For Pre1, Pre2, Post1 and Post2 epochs 2-6 were averaged, whereas for Adapt 1 and Adapt2 epochs 2-11 were averaged. For each kinematic parameter, a repeated-measures ANOVA compared group (G) (sham, cb, m1) and block (B) (6).

FIG. 16 is a tabulation of Kinematic parameters: Experiment two. Movement duration (msecs), reaction time (msecs) and maximum velocity (cm/s) were assessed for the sham, cb and m1 groups in each block of experiment one. Values depict the mean (±SEM) which was determined for each participant by averaging over consecutive epochs. For Pre1, Pre2, epochs 2-6 were averaged. For Adapt 1, Post1, Post2 and Post3 epochs 2-11 were averaged. For each kinematic parameter, a repeated-measures ANOVA compared group (G) (sham, cb, m1) and block (B) (6).

### DESCRIPTION OF THE PREFERRED EMBODIMENT

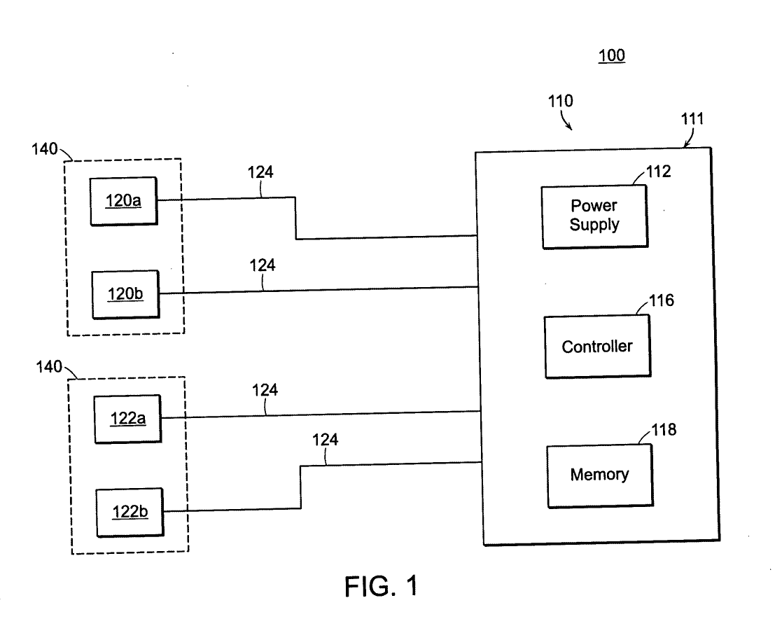

Referring now to the various figures of the drawing wherein like reference characters refer to like parts, there is shown in FIG. 1 a schematic block diagram of an exemplary DC stimulation device 100 according to the present invention. Such a stimulation device 100 includes a DC stimulator 110, a plurality of pair of electrodes 120a,b; 122a,b that are each operably coupled to the DC stimulator 110 via a plurality of cables 124. The DC stimulator 110 includes a power supply 112, a controller 116 and in more particular embodiments a memory 118. The DC stimulator 110 also includes a housing 111 in which the power supply 112, the controller 116 and the memory 118 are stored and a support element 140 to which the electrodes are coupled.

While the stimulation device 100 in the illustrative embodiment in FIG. 1 is depicted as having four electrodes 120a,b; 122a,b or two pairs of electrodes this is not limiting. It is within the scope of the present invention for such a stimulation device to be configured and arranged so as to include two or more electrodes, a single pair of electrodes and two or more pairs of electrodes. Also, while such a device can be configured so as to include one or more pairs of electrodes, it is within the scope of the present invention for a number of electrode pairs less than the total number of available electrode pairs to be utilized at a time.

The electrodes 120a,b; 122a,b are any of a number of electrodes known to those skilled in the art and appropriate for the intended use. In illustrative embodiments, each of such electrodes is a surface electrode having for example a metallic portion and a padded or foam cover. It also is within the scope of the present invention for a material to be applied to the electrode portion in contact with the skin so as to improve the conductive coupling of the electrode to the skin. In exemplary embodiments, such a material is any of a number of gels or liquid solutions known to those skilled in the art for use in improving such skin-electrode coupling conductivity. Similarly, the cables 124 are any of a number of cables that are appropriate for the intended us and for minimizing line losses at the currents and voltages.

The support element 140 includes any of a number of structures known to those skilled in the art, that are adaptable or configurable so as to removably support the electrodes 120a,b; 122a,b. In more particular embodiments, the support element 140 is configured and arranged so it also can be arranged on the head of an individual or person during a learning and training event. In illustrative embodiments, the support element 140 is headgear including but not limited to a cap, hat, head band, or bandana. The headgear or support element 140 provides a mechanism such that the electrodes 120a,b; 122a,b are generally maintained in preselected localization to the head depending on the activity being trained.

Such headgear and support element 140 also is configurable so it and the electrodes can be adjusted to head size or manufactured in different sizes ensuring predetermined electrode position for different head sizes. In yet further embodiments, the support element 140 is configured and arranged so that the electrodes are removably attached to the support element. In this way, the electrodes 120a,b; 122a,b that are mounted on one support element having one configuration or size can be removed and then attached to another support element that is appropriately sized and/or configured for a given use/user.

As described herein, in illustrative embodiments the DC simulator 110 can be an application specific composition of a number of functionalities. It is within the scope of the preset invention to adapt any one of a number of devices known to those skilled in the art that can output DC current and voltages in the desired ranges, so as to function as the DC simulator of the present invention.

The powers source or power supply 112 of the DC stimulator is any of a number of power supplies that can generate the DC current at the desired level(s) and desired voltages. In particular illustrative embodiments, the power supply delivers a desired current so that the DC current at the electrodes is in the range of 0-2 milliamps or 0-4 milliamps. The DC voltage is about 1 millivolt. It should be recognized that these voltages/currents are illustrative and the current and voltage is determined dependent on needs and safety considerations. In further illustrative embodiments, the power supply 112 includes one or more batteries such as for example a conventional nine volt battery and circuitry operably coupled to the one or more batteries so that the current and voltage being outputted by the DC stimulator is in the desired range.

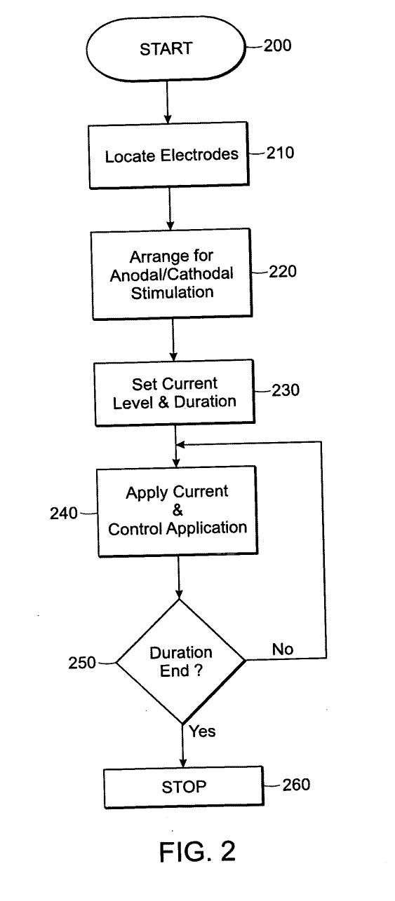

The controller 116 is any of a number of devices known to those skilled in the art (e.g., digital processor, microprocessor, application specific integrated circuit—ASIC) that can carry out or perform the functions as described herein for the controller. Such a DC simulator 110 also can include a memory 118 that is configured so the program or application including the code segments, instructions and criteria for performing the controller functions is stored therein for retrieval when the controller starts up. The controller 116 is operably coupled to the power supply and the cables so that the controller can control which of the pairs of electrodes is being energized, the intensity and duration of the DC current being applied, and the type of DC current electrode stimulation (i.e., anodal or cathodal). The functions of the controller 116 are discussed further below in connection with FIG. 2.

The housing 111 is any of number of structures known to those skilled in the art for housing the power supply, the controller and the memory and preferably is configured and arranged so that the housing can be removably carried by the user at least during a learning or training event. In illustrative embodiments, the stimulator device 110 is configured and arranged so as to portable and attachable to the body of the user using any of a number of different devices including but not limited to a bracelet, belt, waist bags, or placed in clothing pockets or backpacks.

Referring now to FIG. 2 there is shown a high level flow chart or diagram of a method according to the present invention for increasing learning and effects of training in healthy individuals and patients after brain lesions using dc stimulation. Reference also should be made to FIG. 1 and the related discussion for features referred to in the following. This flow chart(s) illustrates the structure of the logic of the different methodologies/inventions, which can be embodied in computer program software for execution on a computer, digital processor or microprocessor. Those skilled in the art will appreciate that the flow chart(s) illustrate the structures of the computer program code elements, including logic circuits on an integrated circuit, that function according to the present inventions. As such, the present inventions can be practiced in its essential embodiments by a machine component that renders the program code elements in a form that instructs a digital processing apparatus (e.g., computer) to perform a sequence of function step(s) corresponding to those shown in the flow diagrams. It also is within the scope of the present invention to be configured so as to perform the functions using hardware.

Such methods of the present invention for increasing learning and the effects of training, utilize DC stimulation of the brain tissue such as with the device 100 described above. Such featured methods increase learning and the effects of training in healthy individuals and/or patients after brain lesions. More particularly, such methods and devices advantageously would improve one's ability to acquire knowledge or skill in such learning and training and/or increase one's ability to retain acquired knowledge or skill in such learning and training. Subjects. persons or patients using such devices and methods can achieve larger results given the same amount of practice when not using such devices or methods. Such would be the case for skill physical activities like typing, playing instruments, sports, etc, and other cognitive tasks, like learning facts, strings or words etc. More particularly, application of such DC stimulation should benefit people practicing or learning sports, how to play musical instruments, dance, hand skills or occupations that require fine motor skills (i.e., typist, dentists, seamstress, assembly line workers, painters, hand artists like sculpture, video game playing, handling or interfacing with robots, machinist, crane operators, mechanics, drivers, pilots, etc). Similarly, patients who suffered neurological lesions and need to perform rehabilitation exercises will benefit further when the training is done under the influence of DC stimulation whether the stimulation is applied immediately before, during or after the completion of the exercises. In addition, DC stimulation also should benefit other activities that are not exclusively motoric in nature, such as language and speech learning, visual coordination and discrimination, activities where perception and sensitivity demands are high, swallowing, and audition.

The method starts (Step 200) by performing those actions in preparation of the execution of the training or learning event actions as well as preparing the individual or patient. For example, if headgear or a support element 140 is to be used in combination with the electrodes, the user or other person would obtain a support element that is appropriately sized and configured for the intended use including a support element that is appropriate for the person (e.g., a hat or cap that is appropriate for a man or woman). Other actions would include applying a gel or liquid solution to the area of the head or the electrode so as to improve coupling conductivity with the skin.

After performing any such initial actions, the method proceeds with localizing or locating the electrodes on the head of the individual or person (Step 220). In other words, the electrodes are positioned on the head so that when the electrodes are energized a DC current of a desired intensity is applied to an area of the brain to specific and/or other parts of the central nervous system dependent upon the skill or task to be trained on/learned so as to improve one's ability to learn or train a given skill or task. That is one area maybe appropriate to stimulate one's ability to learn/train a skill/task but would not be appropriate to improve one's ability to learn or train another given skill or task. Also, stimulating one area of the brain may be appropriate to stimulate one's ability to acquire knowledge or skill whereas another area of the brain may be appropriate for stimulating one's ability to retain acquired knowledge or skill. In particular embodiments, such locating includes locating more than one pair of electrodes on the head at different locations so that when the electrodes are energized, the applied DC current is such as to stimulate one's ability to both acquire knowledge or skill and retain the acquired knowledge.

In yet further embodiments, the electrodes are used in combination with a support element 140 or headgear. Thus, in particular embodiments, such localizing includes arranging the electrodes with respect to the support element 140 such that when the support element is put on the head, the electrodes should be in the appropriate location. For example, if the electrodes are arranged so as to removably secured to a baseball cap, when the individual puts the cap on, the electrodes are appropriately positioned for the desired training/learning event.

Correspondingly, if the one portion of the desired training/learning is directed to a motor skill and another portion of the training/learning is directed to a cognitive task, the cap could be initially configured with one or more pairs of electrodes so as to allow the electrode pair to be energized that would be appropriate for the specific training or learning exercise. Alternatively, the cap could be removed from the head after completing the one portion, the electrodes reconfigured to perform the another portion and the individual could place the cap again on their head.

After locating the electrodes, the electrodes are arranged electrically so that they are configured for one of anodal or cathodal stimulation of the brain tissue (Step 230). In more particular embodiments, the DC stimulator 110 is configured and arranged so that the applied DC current flows in the appropriate direction for such anodal or cathodal stimulation. In yet further embodiments, the electrodes are arranged so that one of the electrodes is applied or secured to a different location of the nervous system (e.g., face chick, shoulder, chin, etc.) so that the effect of stimulation is from one electrode while the opposite electrode (e.g., cathodal) decrease function of another part of the brain. In yet further illustrative embodiments, such configuration can be accomplished without removing the electrodes from the head.

In such methods, the current level or intensity of the DC current is set to a desired level and the duration of the application of such current also is set (Step 230). As indicated herein, the DC current is set at a level that is sufficient to stimulate the area of the brain so as to thereby improve one's to learn a skill or perform a cognitive task during a learning or training event. Such setting of the current and voltage being outputted by the DC stimulator 110 also is set so as to be appropriate for the intended application (improving acquisition of knowledge/skill or improving retention of the acquired knowledge/skill).

As to the duration of the application or delivering of the DC current, it is within the scope of the present invention to apply or deliver such DC current one of before, during, or after the performance of the training or learning event. In yet further embodiments, such application or delivering of the DC current or the direct current stimulation includes (a) delivering direct current stimulation before and during the performance of training (b) delivering direct current stimulation during and after the performance of training; or (c) delivering direct current stimulation before, before and after the performance of training. In yet further embodiments, such application or delivering of direct current stimulation is for a predetermined time before and/or after the performance of training.

After the electrodes have been appropriately located and the current to be applied is set, the process continues with controllably applying the DC current to the electrodes (Step 240), where such controlling includes maintaining a relatively stead desired current. In yet further embodiments, such controllably applying includes ramping up the DC current slowly over the course of a predetermined period of time, (e.g., several seconds) when the current is initially or started to be applied. While this controllable direct current stimulation is ongoing, the method continuously evaluates to determine if the duration has ended or expired (Step 250). In other words, a determination is made as to whether the time period established for applying the direct current stimulation has ended. If the duration or time period has not ended (No, Step 250) then the process continues with controllably applying the DC current to the electrodes (Step 240). If the duration or time period has ended (Yes, Step 250) then the process of controllably applying the DC current to the electrodes (Step 240) is stopped (step 260).

If another training or learning activity or event is planned during which direct current stimulation is to be used to stimulate or improve one's training or learning ability, then the above described steps (steps 210, 220, 230, 240 and 250) are repeated for each additional training or learning activity.

### Example 1

### Introduction

Accurate motor performance is essential to almost everything we do, from typing, to driving, to playing sports. Having a motor skill implies a level of performance in a given task that is only achievable through practice. Evidence indicates that motor skill learning can continue over a prolonged time period. Within-session performance improvements (online effects) occur in the minutes or hours of a single training session and continue over days and weeks of repeated training sessions until performance nears asymptotic levels. Changes in performance can also occur between training sessions (offline effects), i.e., performance at the beginning of session n+1 is different than performance at the end of session n. The use of the term “offline learning” is intentionally avoided herein in connection with Example 1 because it has been used to refer to both a physiological process (consolidation) and a particular measurement result (a positive offline effect). Finally, skills can be retained to varying degrees over weeks to months after the completion of training (long-term retention). Here the effect of noninvasive cortical stimulation on measurements of these three temporal components of skill learning (online effects, offline effects, and long-term retention) were investigated. The principle underlying this approach is that if a perturbation has selective effects on these measures, then this would support the existence of distinct mechanistic processes corresponding to the three temporal components of skill learning.

To examine the effect of tDCS on the different temporal components of motor skill learning, a new sequential visual isometric pinch task (SVIPT) was devised that is sufficiently difficult to ensure that performance continues to improve over five days of training. The difficulty of the task made it comparable to real life skill-requiring tasks, which often take weeks to months to acquire proficiency. In order to quantify skill the speed-accuracy trade-off function (SAF) was empirically derived for the SVIPT. This derivation is important, because otherwise it is not clear how to relate changes in speed and accuracy to a change in skill. It is believed that the formal consideration of changes in the SAF with training is a conceptual advance in the study of skill learning.

For example, if a tennis player hits 125 mph serves but only gets 25% of the balls in the service box, is he more, less, or equally skilled in comparison to a player who hits the ball at 100 mph but gets 50% of the balls in? Answering this question in general requires the ability to distinguish between whether (i) the SAF has changed (which would mean that skill has changed) or (ii) performance is sampled at a different place on the same SAF (which would mean that skill has not changed). Therefore, a skill measure was developed such that a change in it reflected a change in the SAF. It was then determined if anodal tDCS applied over M1 differentially modulates online effects, offline effects, and long-term retention.

### Results

Characterization of the Speed-Accuracy Trade-Off Function (SAF) for the SVIPT and Derivation of a Skill Measure

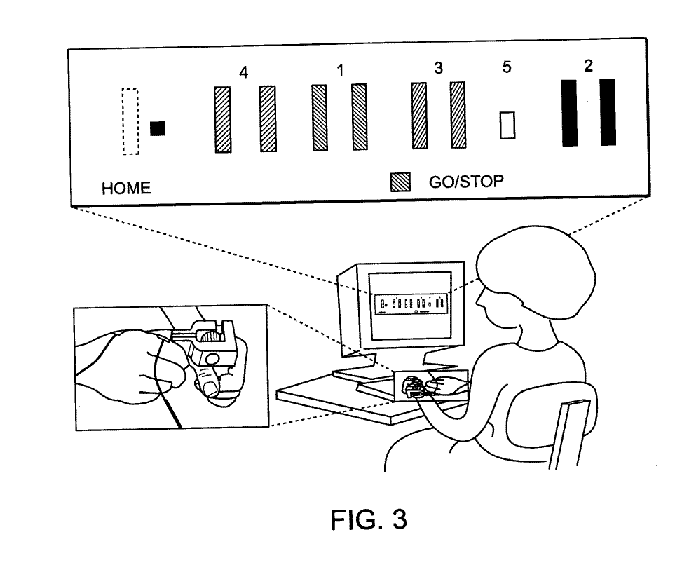

The SVIPT is shown in FIG. 3. Speed was captured by the average sequence movement time per training block (movement onset to stopping at gate 5). Accuracy was defined as the proportion of trials per block with hits to all 5 targets in the correct sequence order. The error rate was calculated as [1—accuracy rate]. Skill was defined as a practice-induced change in the SAF. It was first established that the SAF of the SVIPT does indeed change over 5 days of training.

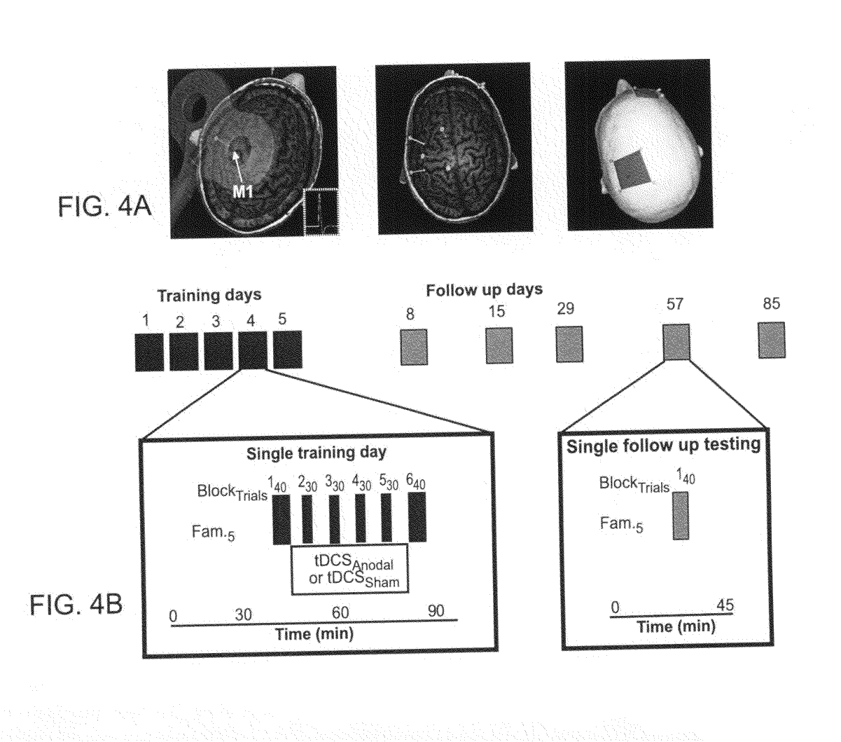

The SAF was estimated in a sample of subjects who were either naïve to the task (n=12, age 32±3 years, 7 M), or were trained on the task over a 5 day period (n=6, age 28.3±2.6 years, 3 M). The 5 day training schedule was identical to that in the main experiment as shown in FIGS. 4 A,B, but without tDCS.

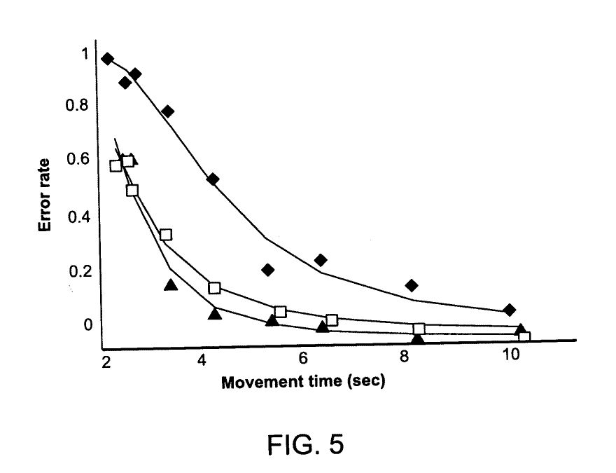

To estimate the SAF, every subject performed 9 blocks of 10 trials each, with each block set at a different movement time (MT) imposed by a metronome. The order of the blocks was randomized and balanced within each group. In the naïve group, MTs averaged 10.1±0.1, 8.2±0.1, 6.4±0.06, 5.3±0.04, 4.3±0.06, 3.4±0.1, 2.8±0.08, 2.6±0.03, and 2.3±0.03 seconds. For the trained group the MTs averaged 10.3±0.28, 8.2±0.08, 6.4±0.06, 5.4±0.06, 4.3±0.08, 3.4±0.12, 2.7±0.08, 2.6±0.04, and 2.4±0.06 seconds. There was a clear difference in the SAF between groups, with a given MT tending to be associated with a higher accuracy in the trained group compared to the naïve group (FIG. 5). A two-parameter model was empirically chosen that fit these SAF data well and for which only one of the parameters (the so-called “skill parameter”; the larger the value of the parameter, the higher the skill) substantially changed with training. As one fit many functions to the pre- and post-training SAFs, there was the possibility of a spuriously good fit. Therefore, this model was validated by acquiring another SAF dataset in a separate set of trained subjects (n=6, age 29±1.9 years, 2 M). For those subjects average MTs were 10.3±0.14, 8.3±0.08, 6.6±0.06, 5.5±0.04, 4.3±0.07, 3.3±0.05, 2.7±0.06, 2.6±0.06, and 2.4±0.07 seconds. The model fit this SAF well, and moreover there was a near-perfect overlap of the SAFs for the original and validation groups as shown in FIG. 5.

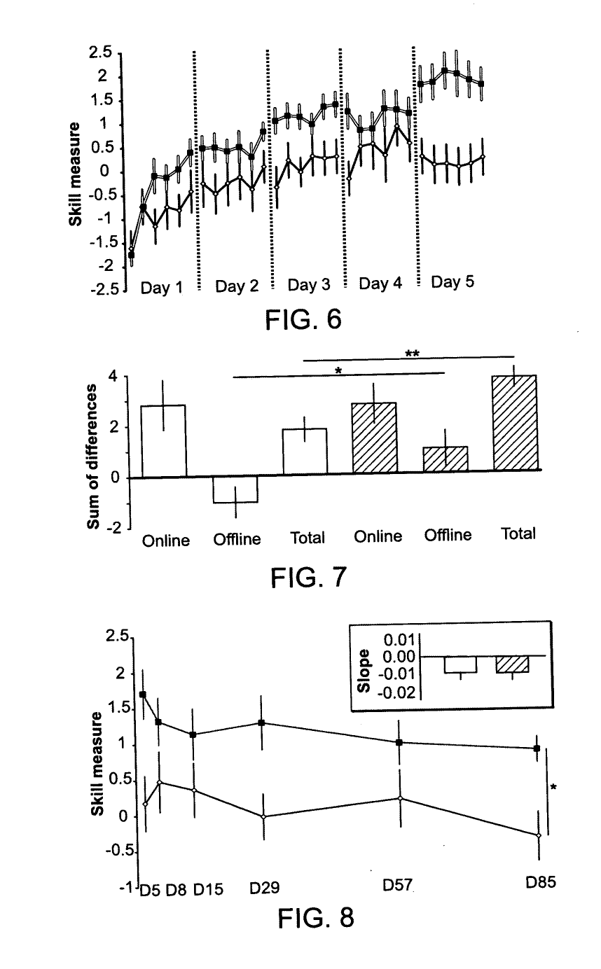

Having validated the SAF model, one would be justified in estimating the skill parameter corresponding to each bivariate observation (per subject and per block) of error rate and movement time. It was found that variability in the estimated skill parameter was multiplicative (data not shown). In order to validly perform parametric statistical analyses, the skill parameter was logarithmically transformed to homogenize the variances (see FIG. 6). The natural logarithm of the skill parameter is referred to in the remainder of this report as the “skill measure”, and is used exclusively in all statistical analyses. The effects of time on the skill measure were decomposed into online (within-day) and offline (between-day) effects. The latter are thought to reflect consolidation. Total learning equaled online effects plus offline effects.

Application of Anodal tDCS Over the Primary Motor Cortex Enhanced Skill Acquisition

Having established in that training on the SVIPT increases skill, it was then determined whether anodal tDCS applied synchronously with motor training enhances the total amount of skill acquired over 5 days of training in comparison to sham tDCS. Two groups of subjects underwent daily training with the SVIPT (FIG. 1) for 5 days (200 trials per day, organized into 6 blocks (40-30-30-30-30-40 trials) under anodal tDCS (n=12, age 28.3±2.2 years, 5 m, 7 f) or sham tDCS (n=12, age 30.8±3.0 years, 7 m, 5 f), see FIG. 2.

The average skill measure for the anodal tDCS and sham tDCS groups is plotted versus training block in FIG. 6. Both groups started with the same level of skill in the first block of day 1 [p=0.8199]. As expected, the sham tDCS group exhibited positive total skill learning over the training course [p=0.002]. The anodal tDCS group exhibited greater total learning than the sham tDCS group [p=0.005].

To determine whether the learning-enhancing effect of tDCS was polarity specific, a separate group of 12 subjects (age 28.3±1.4 years, 6 m, 6 f) was trained over 5 days with synchronously applied cathodal tDCS. There was no significant difference in total learning between the cathodal and the sham tDCS groups [p=0.494], but there was a significant difference in total learning between cathodal and anodal tDCS [p=0.0055]. Thus, the observed effect of tDCS on skill acquisition is indeed polarity-specific.

Anodal tDCS Enhanced Acquisition Through Positive Offline Effects

Having established a difference in total learning between anodal tDCS and control conditions, it was next investigated how anodal tDCS alters online effects and offline effects. There was no significant difference in online effects between the two groups [p=0.954] (see FIG. 7), but a significant difference in offline effects [p=0.04] (FIG. 5). In fact, the sham group showed offline forgetting [difference from zero: p=0.05], as has been described in other skill learning studies. In contrast, the anodal tDCS group showed a trend toward positive offline effects [difference from zero: p=0.091]. Therefore, as total learning is simply the sum of online and offline effects, the improved total learning in the anodal tDCS group appears to be driven by the positive offline effect.

One potential concern in interpreting this difference in offline effects between sham and anodal tDCS groups is that the first 40 trials per day were used to calculate the offline effect (see METHODS). It is possible that the apparent positive offline effect in the anodal tDCS group was due to an enhancement of learning rate within the first 40-trial block of each day. This possibility was ruled out by assessing offline effects with only 5 trials. There was a significant difference for offline effects between the anodal tDCS and sham tDCS groups using this higher resolution offline measure [p=0.00065], with the anodal tDCS group showing a significant positive offline effect [p=0.00048] and the sham tDCS group showing a significant negative offline effect [p=0.019]. We conclude that anodal tDCS applied synchronously with training induces offline gains, in contrast to an offline loss seen in the sham tDCS group.

Anodal tDCS Did not Enhance Long-Term Retention

Skill retention was evaluated at 5 time points with a single testing block of 40 trials at days 8, 15±1, 29±2, 57±2 and 85±2 (see FIG. 4B and FIG. 8). The two groups forgot at the same rate even though they started from different levels of skill on day 5 (see FIG. 8). A t-test comparing the slopes of forgetting across the 85 days between conditions showed no significant difference [p=0.971] (see insert FIG. 8). The persistence of greater skill in the anodal tDCS group compared to the sham tDCS group at all recall time points indicates the robustness of the tDCS effect. The skill measure was still significantly higher in the anodal tDCS group compared to the sham group at Day 85 [p=0.005]. It thus was concluded that anodal tDCS increased total skill learning over the 5 training days, but did not change the rate of forgetting after training.

### DISCUSSION

The effect of anodal tDCS applied over M1 on motor skill acquisition over 5 consecutive days and its retention at several time points over a three-month follow-up period were investigated. There were three main findings. First, anodal tDCS in combination with training led to significantly greater total learning at the end of 5 days compared to sham. Second, the greater total learning in the anodal tDCS group was primarily mediated through induction of positive offline effects. Third, anodal tDCS did not affect the rate of forgetting over the 3-month follow-up period. The greater total learning in the anodal tDCS group at the end of day 5, however, meant that skill remained superior in this group compared to sham tDCS at 3 months.

### Skill

Previous studies of skill learning have reported speed and accuracy measures separately. As explained in the introduction, using parallel measures can lead to ambiguity if they change in opposite directions or if changes are subtle. Here skill acquisition is formally defined as a change in the SAF. First one derived a skill measure based on empirical estimations of the SAF in a separate group of subjects. It then was possible to use this skill measure for the main tDCS experiment without needing to generate SAFs at each time point.

The task used in this study was designed to assess skill rather than adaptation. Adaptation allows the motor system to regain former levels of performance in the setting of a perturbation, whereas skill is the acquisition of a higher level of performance. Within the computational framework of optimal feedback control it could be posited that adaptation is mediated through changes in a forward model, whereas skill represents a slower process of acquiring an optimal feedback control policy. This difference is apparent in the respective time course for these two kinds of motor learning. Adaptation to a perturbation—whether it is to prisms, vasomotor rotations, or force fields—reaches asymptote within one day.

In contrast, skills take much longer to acquire. In this context, the SVIPT a skill task was considered because subjects improve their level of performance, reflected in a new SAF, over a prolonged period of time, and therefore it shares psychophysical similarities with other skills such as sequential finger tapping or piano playing rather than with adaptation tasks or the Serial Reaction Time Task (SRTT), which measures acquisition of sequence order rather than performance accuracy. The distinction between adaptation and skill is particularly important in the context of brain stimulation and localization because they appear to be mediated by separate neural substrates. For example, finger-tapping skill tasks typically show learning-related activation in contralateral M1, whereas adaptation tasks, such as visuomotor rotation, predominantly activate posterior parietal cortex and cerebellum.

### Online Skill Acquisition

Three previous studies have shown enhancement of motor learning in a single session using anodal tDCS in healthy subjects. Although the a priori hypotheses focused on net learning effects across 5 days of training, visual inspection of FIG. 6 suggests that there was indeed a greater day 1 within-session effect for anodal tDCS, consistent with these previous reports, supporting the idea that the pre-existing synaptic machinery is strengthened by tDCS. On the other hand, over the course of 5 training days there was no significant online effect for anodal tDCS compared to sham. These results suggest that the neural substrates underlying online effects, which are likely to include LTP-like mechanisms, may become saturated early on, manifesting as a ceiling for behavioral improvements within-session. Consistent with this interpretation, it has been shown that repeated motor training in rats led to occlusion of LTP expression in the motor cortex, typically paralleled by a ceiling in reaching skill gains.

One might expect that polarity reversal would yield the opposite behavioral pattern (reduced consolidation). However, cathodal tDCS did not influence the learning process relative to sham, a result consistent with previous findings on day 1 using a different task. Such results, in combination with the known down-regulating effect of cathodal tDCS on motor-cortical excitability, suggest caution when trying to predict behavioral consequences of brain stimulation based on effects on motor cortical excitability.

### Offline Consolidation

Motor consolidation is understood to mean either positive offline effects (“offline learning”) or resistance to interference. Positive offline effects mediated the greater total learning in the anodal tDCS group: on average, performance at the beginning of day n+1 was better than at the end of day n. The robustness of this offline effect was apparent in the fact that it was also present whether derived from the initial 5 trials or 40 trials, arguing against an enhanced practice effect (savings) within the first training block. In contrast, the sham group showed an offline loss in skill.

A decrease in performance after a rest period is well described for skill learning and has been called the “warm-up decrement.” As saw in the sham group, the warm-up decrement is small, in so much that it does not reduce performance to naïve levels. Positive offline effects, which were observed only in the anodal tDCS group, are thus not a ubiquitous phenomenon in skill acquisition. Nevertheless, positive offline effects have garnered a great deal of attention in recent years as they have been reported in several influential studies of skill consolidation with finger-sequencing tasks. To the best of one's knowledge, there has been little comment about why some skill tasks show a warm-up decrement whereas others show consolidation. One possible explanation may relate to the distinction between continuous and discrete skill tasks. In a continuous task, as in this study, the behavior continues in an uninterrupted fashion during each trial.

In contrast, finger-sequencing tasks, such as the SRTT, consist of separable discrete finger movements. Warm-up might not be needed in the latter case as one can explicitly call up each discrete element (finger movement). Support for a role of an explicit component in offline gains comes from the observation that sleep-dependent offline learning occurs for the SRTT, when subjects acquire explicit awareness of the sequence during training. Here a novel finding is presented, that tDCS induces consolidation in a continuous skill task, where an overnight decrement is the default occurrence (sham group). This finding implies that for a continuous skill the passage of time and/or sleep may be necessary but not sufficient for consolidation to occur. One cannot answer whether tDCS could induce consolidation in the absence of sleep because a within-day, two-session experiment was not performed.

What could the mechanism of tDCS-induced consolidation be? The effect of anodal tDCS on cortical excitability is known to outlast the stimulation period. This effect has been documented as increased firing rate of single cortical neurons in rats and as increased motor evoked potentials (MEPs) as measured by TMS in human studies. Animal data suggest that skill learning after day 1 depends on plasticity-related protein synthesis. Thus, one can postulate two possible mechanisms for anodal tDCS-induced positive offline learning: either anodal tDCS enhances protein synthesis directly during training or its excitability effects during and after training interact downstream of learning-related protein synthesis. Further work in animals and slice preparations will be needed to address these alternative mechanisms.

### Long-Term Retention

Anodal tDCS did not have any effect on the rate of forgetting over the ensuing three months relative to sham tDCS. The equal rate of forgetting seen in the two groups led to a higher skill level at day 85 in the anodal tDCS group. This finding is important, because a potential cost of faster learning over 5 days could have been faster forgetting. If there is an evolutionary reason why maximal potential levels of learning are not reached in the absence of stimulation, then there could be a hidden cost to learning enhancement that we do not currently appreciate. The finding that anodal tDCS induced offline consolidation but did not hinder the rate of forgetting also suggests that the overnight warm-up decrement and forgetting of skill over weeks and months are distinct processes. Alternatively, the possibility that the warm-up decrement seen in the sham group was not affected per se but over-ridden by an independent consolidation effect of anodal tDCS cannot be ruled out.

Role of M1 in Motor Learning

In concordance with previous reports, the results suggest that M1 is a key structure in motor skill learning that can be purposefully modulated by noninvasive brain stimulation. That its role in the consolidation of motor skills can dissociate from the initial acquisition was shown by a study in which low frequency repetitive TMS was applied over M1 between same-day training blocks of a ballistic pinch task. rTMS disrupted short-term retention of performance 15 min later, but not online learning or subsequent retention if applied 6 hours after the end of training.

The results herein are consistent with this finding: anodal tDCS had, except for day 1, minimal effects on within-day acquisition and on long-term retention but had a marked effect on consolidation. Hence, decreasing motor cortical activity (using 1 Hz rTMS in) and enhancing it (using anodal tDCS in this study) may have opposite effects on consolidation. It is therefore conceivable that M1 is involved in early consolidation of motor skills and that this consolidation can be enhanced or disrupted by non-invasive cortical stimulation either through a direct effect on M1 or indirectly through effects on other motor regions connected with M1. Whether there is a categorical difference when the between-session period includes sleep, as recently suggested by a study using rTMS and a variant of the Serial Reaction Time Task, will require future investigation.

### CONCLUSIONS

The finding that the effect of anodal tDCS was specific for induction of consolidation, as opposed to enhancement of online effects or long-term retention, supports the view that motor skill learning comprises mechanistically and temporally distinct processes. The persistence of a beneficial effect of anodal tDCS at three months after the end of training may have promising implications for the design of motor learning protocols in healthy individuals as well as in patients undergoing neurorehabilitation.

### Materials and Methods

### General

All subjects gave written informed consent to participate in the study according to the declaration of Helsinki The study was approved by the NINDS Institutional Review Board. Experiments were carried out in the Human Cortical Physiology Section, NINDS, NIH. Participation required a normal general and neurological examination, dominant handedness, lack of chronic neurological or psychiatric disease or any severe medical condition, and lack of drug intake.

### Training and Follow Up

All subjects underwent ˜45 min of repeated task practice (200 trials separated into 6 blocks on 5 consecutive days (see FIGS. 4 A,B). Sessions took place between 8 am and 2 pm and were separated by 24 hrs (start-start). Subjects received either sham tDCS or anodal tDCS for 20 minutes during training (see FIG. 4B). Training block 1 and 6 was always performed without stimulation. A third group served as active control in a separate experiment receiving cathodal tDCS during training.

Retention of skill (40 trials of the SVIPT) was tested on Day 8, Day 15±1, Day 29±2, Day 57±2 and Day 85±2 (see FIGS. 4B and 8). All retests were performed at the same time of the day and in the same experimental environment as the training sessions.

### Transcranial Direct Current Stimulation

tDCS was applied via two conducting 25 cm² electrodes covered by a saline-soaked sponge. A bipolar electrode montage (left motor cortex and right supraorbital area) was used (see FIG. 4A). The terminology “anodal” and “cathodal” refers to the electrode placed over the left M1. The M1 hand area was localized in all subjects with transcranial magnetic stimulation and in addition, in a subgroup of volunteers, using a neuronavigation device (see FIG. 4A). A Phoresor® II Auto (Model PM850, IOMED®, Salt Lake City, Utah, USA) device was used to apply tDCS. The stimulation was delivered at an intensity of 1 mA (current density 0.04 mA/cm2; total charge 0.048 C/cm²) for 20 minutes in the anodal and cathodal tDCS groups, and for up to 30 seconds in the Sham session according to a previously described method. Subjects and the investigator performing motor testing and data analysis were blinded to the type of tDCS.

### Sequential Visual Isometric Pinch Task (SVIPT)

Subjects were seated in an armchair 60 cm in front of a 20 inch-screen monitor. Subjects held a force transducer between the thumb and the index finger of the right hand. Squeezing the force transducer moved a screen cursor horizontally to the right, while relaxing caused the cursor to move left. Upon presentation of a GO signal, the goal of the task was to move the cursor quickly and accurately between the start position (Home) and a numbered order of gates (Home-1-Home-2-Home-3-Home-4-Home-5). A STOP signal appeared when stopping at gate 5. To increase the difficulty of the task, a logarithmic transduction of pinch force into cursor movement was chosen with the maximum rightwards movement set to 35-45% of maximum force. The average movement time per training block was measured from movement onset to stopping at gate 5. The error rate was calculated as the proportion of trials with at least one over- or undershooting movement.

### Psychophysical Assessment

During all sessions, subjects underwent a brief psychophysical assessment and were asked to report all potential side effects.

### Determination of Skill

### Skill Parameter

The elementary notion guiding our definition of skill was that a change in skill is equivalent to a change in the SAF (see FIG. 5). Therefore, behavior reflecting a change in position along the curve of an unchanged SAF should not be interpreted as an authentic change in skill. To define a measure of skill that satisfies this notion, one conceivable approach would be to estimate the complete SAF in every subject at every time point during training. As such an approach is not practicable, an alternative method was chosen. The goal was to develop a parsimonious mathematical model for the SAF of the SVIPT such that training (which was confirmed to indeed change the SAF, see FIG. 5) is associated with a selective change in one model parameter, which could therefore define as the skill parameter. It is reasoned that if one could find such a model, then by using fixed estimates of the non-skill parameters, one would be able to estimate the skill parameter in each subject and time point during training simply from their corresponding bivariate observation of speed and accuracy.

Using a model that was validated in an independent sample of subjects, the proposed estimate of the skill parameter, a, was chosen to be:

$$a = \frac{1 - error rate}{error rate ( {ln ( duration )}^{b} )} ,$$

where error rate and duration (movement time) are averages over some number of trials.

### Multiplicative Model of Learning

The noise in the skill parameter estimate was multiplicative, which required us to logarithmically transform it: the natural logarithm of the skill parameter was called the skill measure. Additive differences between skill measures at two different time points are proportional to the multiplicative difference (i.e., the ratio) of the skill parameters at those two time points. The online effects, offline effects, and total learning across the 5 days of training were then defined as

$$\begin{matrix}online effects := {\sum }_{i = 1}^{5} ( \begin{matrix}skill {measure}_{day i , last block} - \\ skill {measure}_{day i , first block}\end{matrix} ) \\ = {\sum }_{i = 1}^{5} ln ( \frac{{a}_{day i , last block}}{{a}_{day i , first block}} ) = ln ( {\prod }_{i = 1}^{5} \frac{{a}_{day i , last block}}{{a}_{day i , first block}} ) , \\ offline effects := {\sum }_{i = 1}^{5} ( \begin{matrix}skill {measure}_{day i + 1 , first block} - \\ skill {measure}_{day i , last block}\end{matrix} ) \\ = {\sum }_{i = 1}^{4} ln ( \frac{{a}_{day i + 1 , first block}}{{a}_{day i , last block}} ) = ln ( {\prod }_{i = 1}^{4} \frac{{a}_{day i + 1 , first block}}{{a}_{day i , last block}} ) ,\end{matrix} \\ total learning over 5 days of training := online gains + offline gains \\ = ln ( \frac{{a}_{day 5 , last block}}{{a}_{day 1 , first block}} ) .$$

### Statistical Analysis

All data distributions were tested for normality (Kolmogorov-Smirnov test) before choosing parametrical statistical tests. Group differences were assessed by two-tailed t-test comparisons of baseline performance, total learning, online effects, offline effects retention slope and skill at day 85 across groups. Bonferroni-Holm adjustment was used, if more than 2 comparisons were performed. Significance level was set to p=0.05.

### Example 2

### Introduction

The ability of the motor system to adapt to internal (own body) or external (the environment) changes is of fundamental importance to perform accurate movements (Tseng et al., 2007). Motor adaptation refers to situations where, in order to return to a former level of performance, an error stemming from the altered internal or external condition is reduced (Krakauer et al., 2006). Adaptation to altered external conditions has been studied through the application of a screen-cursor transformation during reaching or pointing movements (visuomotor adaptation). This causes a systematic directional bias around the hand and thus can be used to probe adaptive processes (Diedrichsen et al., 2005; Hadipour-Niktarash et al., 2007; Krakauer, 2009; Krakauer et al., 2000; Miall et al., 2004). In fact, visuomotor adaptation has revealed important principles which are thought to be generalisable to procedural learning and memory (Krakauer, 2009).

Visuomotor adaptation, characterized by reduction in reaching errors, is believed to be driven by a mismatch between the predicted and actual sensory outcome of a reaching movement (Tanaka et al., 2009; Tseng et al., 2007). Neuropsychological studies have suggested that the successful reduction of errors during adaptation is a cerebellar-dependent process. For example, patients with lesions in the cerebellum are either unable or heavily impaired in their ability to adapt to changes in visuomotor alignment (Weiner et al., 1983; Martin et al., 1996; Rabe et al., 2009).

Additionally, fMRI studies have associated activation in the cerebellum with visuomotor adaptation (Diedrichsen et al., 2005; Flament et al., 1996; Krakauer et al., 2004; Imamizu et al., 2000). In contrast, other studies have suggested that M1 is involved in the retention of the newly formed motor commands after exposure to adaptation paradigms. For instance, Hadipour-Niktarash et al., (2007) disrupted M1 with transcranial magnetic stimulation (TMS) and found impaired retention but not acquisition of a novel visuomotor transformation. This suggests that the cerebellum and M1 have distinct roles in visuomotor adaptation. Specifically the cerebellum is essential for the reduction of error during acquisition whereas the M1 is important for retention (Tanaka et al., 2009).

Although previous studies have suggested separate roles for the cerebellum and M1 during adaptation, no study has investigated this issue in a direct manner using the same motor task and intervention. In addition, prior studies assessed this by testing patient populations or healthy individuals with virtual TMS lesions, leaving the possibility that some of the changes observed, or the lack of them, were due to compensation by other neural regions. Here it is sought to double-dissociate the roles of the cerebellum and M1 through the application of anodal transcranial direct current stimulation (tDCS), a non-invasive form of electrical stimulation that has been shown to increase M1 (Nitsche et al., 2003) and cerebellar (Galea et al., 2009) excitability. It is hypothesized that cerebellar anodal tDCS would specifically enhance adaptation through a faster reduction in error. In contrast M1 anodal tDCS would augment retention, as shown by increased error during deadaptation.

### Method

### Subject

Sixty right-handed healthy individuals with no history of neurological or psychiatric conditions (26 women; mean age 26±6 years, range 19-41 years) participated in the study. All subjects signed informed consent approved by Johns Hopkins Institutional Review Board and in accordance to the declaration of Helsinki. At the end of each session, participants reported their attention, fatigue and perceived pain of tDCS using a self-scored visual analog scale in which 1 represented poorest attention, maximal fatigue and pain and 7 represented maximal attention and least fatigue and pain (Table 1; Stefan et al., 2005).

### Experiment 1

### Experimental Procedure

Participants (n=30; 14 women; mean age 25±5 years, range 19-40 years) were seated approximately 60 cm in front of a 48 cm diameter computer monitor (with 1280×1024 pixel resolution). The subjects were instructed to move a digitizing pen with their right hand over a horizontal digitizing tablet (62 cm×46 cm; Intuos4, Wacom, USA) located at waist height to reach 8 different targets projected over a computer screen. The position of the pen was sampled at 75 Hz through a custom Matlab program (The MathWorks, MA, USA), which controlled a circular green cursor (2 mm diameter) on a black screen.

Following a well-described protocol (Hadipour-Niktarash et al., 2007), participants performed rapid ‘shooting’ reaching movements to 2 mm diameter targets displayed in one of eight positions arrayed radially at 10 cm from a central starting position. In this manner, subjects moved the cursor from a white square (3 mm diameter) centered in the middle of the screen to the center of the visible target in a straight line. There was a 1:1 mapping between cursor and hand displacement. At the moment the cursor passed through the invisible boundary circle (trial end), the cursor was hidden, the boundary point (endpoint) was marked with a yellow square and when needed a high- or low-pitched tone informed the participant that their movement was either too fast or too slow respectively (275-375 ms). If the movement was within this time window no audio feedback was given.

After each movement participants moved the cursor back to the starting position, however the cursor indicating their hand position only reappeared when they were within 2 cm of the starting position. The targets were presented pseudorandomly so that every set of eight consecutive trials included one of each of the target positions. The pseudorandom order was maintained across participants. During two blocks a 30° counter clockwise (CCW) screen-cursor (visuomotor) transformation was imposed unexpectedly. To ensure their arm and hand was not visible throughout the study participants wore goggles with horizontal blinkers.

Transcranial Direct Current Stimulation (tDCS)

Transcranial direct current stimulation (tDCS) was delivered through two sponge electrodes (surface area: 25 cm²) embedded in a saline-soaked solution. There were three groups (n=10) each with different electrode placements. For the cerebellar tDCS group (cb) the anodal electrode was centered on the right cerebellar cortex, 3 cm lateral to the inion and the cathodal electrode was positioned on the right buccinator muscle (Galea et al., 2009). The primary motor cortex tDCS group (m1) had the anodal electrode placed over the left motor ‘hotspot’, identified by single pulses of transcranial magnetic stimulation (TMS) delivered at a slightly suprathreshold stimulus intensity to elicit responses on the first dorsal interosseus muscle (FDI). The cathodal electrode was placed on the skin overlying the contralateral supraorbital region (Nitsche and Paulus, 2000).

The sham tDCS group consisted of both these electrode positions chosen at random. Anodal stimulation was set at 2 mA (Ferrucci et al., 2008; Galea et al., 2009; Iyer et al., 2005) and was delivered using a Phoresor II Auto (Model No. PM850; IOMED). Thus, a current at a density of 0.08 mA/cm² was applied, which is considered to be safe (Iyer et al., 2005) and is far below the threshold for tissue damage (Boggio et al., 2006). For the sham group, anodal tDCS was applied for 30 seconds. At the onset of tDCS, the current was increased in a ramp-like fashion, a method shown to achieve a good level of blinding (Gandiga, 2006). The experimenter and participant were blinded as to whether anodal or sham tDCS was being applied.

### Experimental Protocol

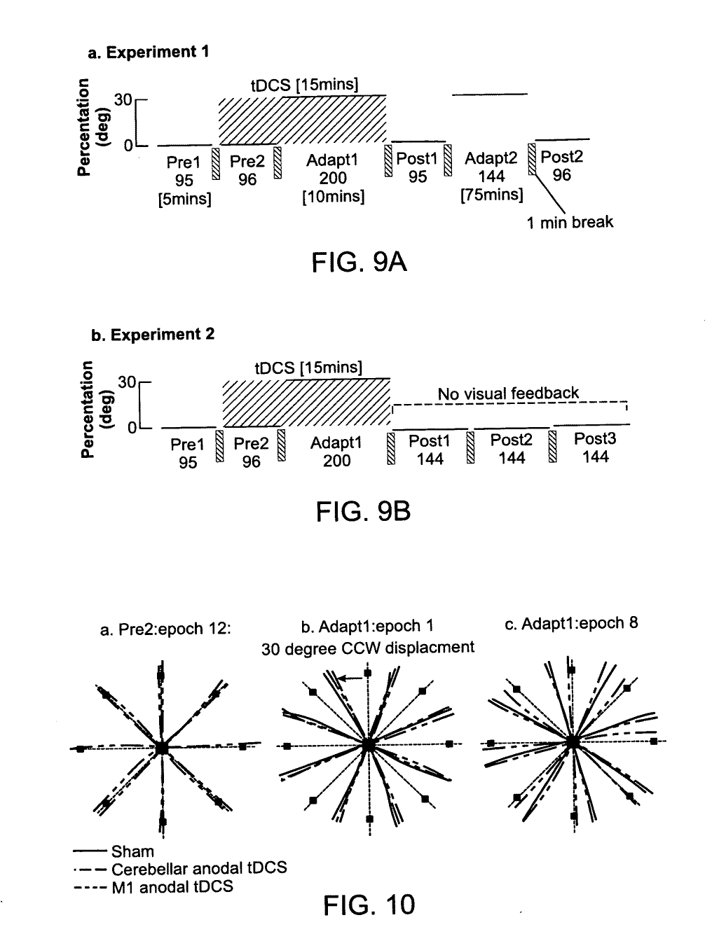

All groups experienced the same experimental protocol consisting of six blocks with one-minute rest periods between blocks (FIG. 9A). Blocks 1 (Pre1), 2 (Pre2), 4 (Post1) and 5 (Post2) involved 12 repetitions of the 8 targets (96 trials) under veridical conditions. The third block (Adapt1) consisted of 25 repetitions (200 trials) where a 30° CCW visuomotor transformation was applied to the cursor on the screen. The fifth block (Adapt2) also involved the same CCW transformation, however only 18 repetitions (144 trials) were performed. Therefore, acquisition was assessed during Adapt1 and 2, and retention with Post1 and 2. Anodal tDCS was applied during Pre2 and Adapt1 (approx 15 minutes; FIG. 9A). During these two blocks the groups differed either in terms of the position of the tDCS electrodes (cb, m1) or the amount of stimulation they received (sham).

### Experiment 2

### Experimental Protocol

During deadaptation (Post blocks), at least two factors influence the error in reaching movements. With every trial, the participant forgets some of what had previously been acquired, reflecting retention (Smith et al., 2006), and simultaneously learns from the movement error (Hadipour-Niktarash et al., 2007). Therefore, to assess retention alone a new group of subjects was exposed to Experiment 2, where the deadaptation trials were performed without visual feedback (Post1, 2, 3; FIG. 9B). Given that participants could not observe movement errors, one was able to assess whether tDCS over the cerebellum or M1 specifically influenced the rate of retention.

Three groups (n=30; 12 women; mean age 27±6 years, range 19-41 years) were exposed to the same experimental procedures used in experiment 1 (sham, cb, m1; see ‘Transcranial direct current stimulation (tDCS)’ section for an explanation of the tDCS electrode placement and amount of stimulation). The first three blocks (Pre1, 2, Adapt1) were identical to experiment 1 with tDCS being applied during Pre2 and Adapt1. Blocks 3-6 (Post 1, 2, 3) consisted of 18 repetitions each (144 trials) in which participants made ‘shooting’ reaching movements without visual feedback (FIG. 9B). During these trials, the target was visible however the cursor indicating the participant's hand position was not. In addition, the participant's did not receive end-point feedback. Subjects were instructed that the square (starting position) in the middle of the screen would turn red once they had passed the target. Audio feedback was still given regarding movement time.

### Data Collection and Analysis (all Experiments)

The 2-D position of the hand was continuously recorded at a rate of 75 Hz using a custom Matlab program (Mathworks, Matick, Mass.). All kinematic data were filtered at 10 Hz with a low-pass Butterworth filter and numerically differentiated to calculate velocity. The onset of each movement was determined as the point at which radial velocity crossed 5% of peak velocity. Performance was quantified in each trial using angular endpoint error, defined as the angle between the line connecting the starting position to the center of the target, and the line connecting the starting position to the endpoint (Hadipour-Niktarash et al., 2007). Positive values indicated CCW error whereas negative values indicated clockwise (CW) error. Epochs were created by binning 8 consecutive movements.

For each block, the initial amount of error (mean error) was determined by averaging over consecutive epochs (Krakauer et al., 2005). For blocks consisting of 96 trials, epochs 2-6 were averaged, whereas for blocks with either 144 or 200 trials, epochs 2-11 were averaged (Krakauer et al., 2005). Using mean error as the primary outcome measure, separate repeated-measures ANOVAs (ANOVArm) were used for each experiment to compare factors group (sham, cb, m1) and block (6). If an interaction was found, separate one-way ANOVAs compared group in each block. Tukey post-hoc tests were performed on all significant results. In addition, identical analysis was performed on (mean) movement duration, reaction time and maximum velocity. All data presented represents mean±SEM unless otherwise specified.

### Results

### Experiment One: Summary

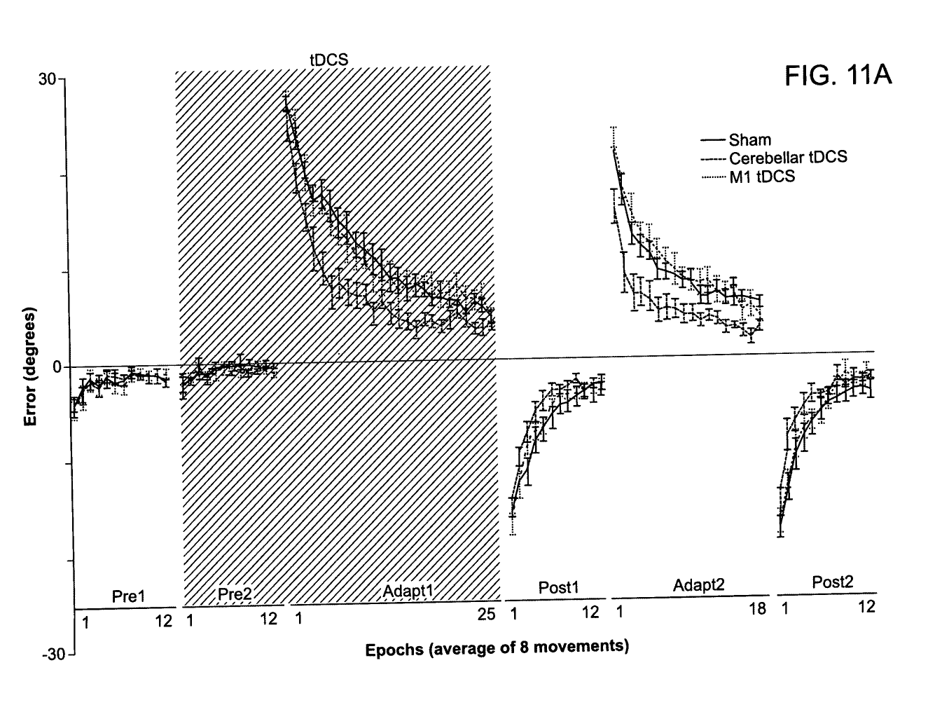

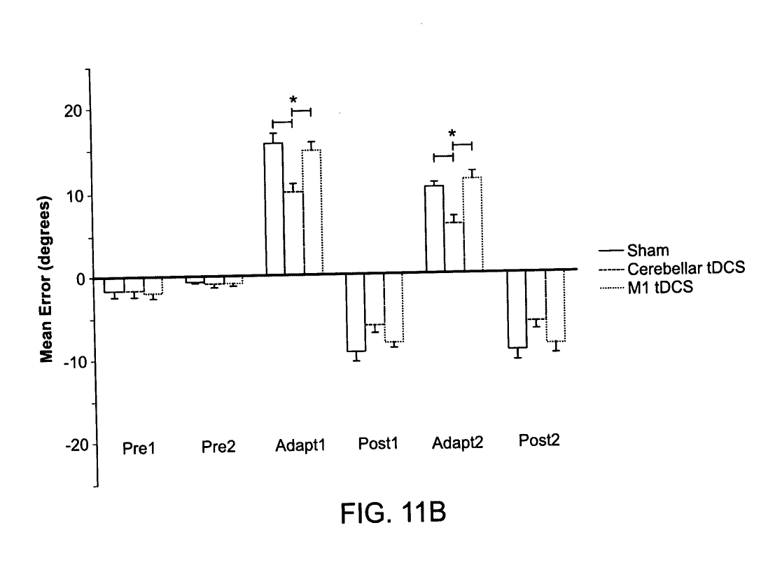

All subjects completed the study without adverse events. In all 3 groups the Pre blocks were characterized by relatively accurate performance with hand trajectories indistinguishable between groups (FIG. 10A). Due to the novel visuomotor transformation a large error in movement trajectory was initially observed in Adapt1 for all groups (FIG. 10B). Over subsequent trials all participants adapted, reducing the error values and returning towards baseline performance. However, during adaptation the cb group showed a smaller amount of error in comparison to the sham and m1 groups (FIG. 10C). This faster reduction in angular error was also observed when plotting its evolution over the course of epochs (FIG. 11A).

Cerebellar tDCS Improves Error Reduction

An ANOVArm, which compared mean error across group (sham, cb, m1) and block (6), revealed no significance difference for group (F[2,27]=1; p=0.3), however there was a significant main effect of block (F[5,135]=293; p=0.0005) and group x block interaction (F[10,135)=6; p=0.0005). Therefore, separate one-way ANOVAs compared group in each block. During Adapt 1, there was a significant effect of group (F[2,29]=8; p=0.002; FIG. 11B). Tukey post-hoc tests revealed a significant difference between the cb (9.7±1.2) group and both the sham (15.7±1.1; p=0.003) and m1 (14.9±1; p=0.01) groups. This indicates that the cb group experienced the largest reduction of error during adaptation (FIG. 11A). In addition, a similar difference between groups was observed for Adapt2 (F[2,29]=9; p=0.001; FIG. 11B), where the cb (5.6±1.1) group showed a greater reduction of error in comparison to either the sham (10.1±0.7; p=0.009) and m1 groups (11.2±1.1; p=0.001; FIG. 11A).

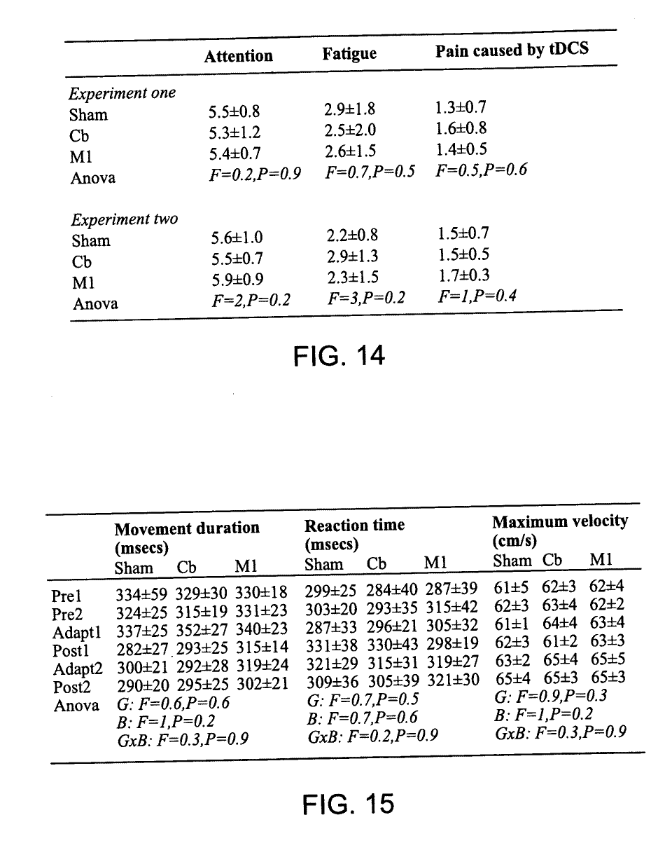

This improvement in error reduction could not be explained by differences in baseline performance, in psychological measures or in movement kinematics. Indeed, although all groups showed a small CW bias at Pre 1 and Pre 2, there was no significant effect of group (sham:−1.6±0.9 degrees, cb:−1.8±0.7, m1:−2±0.8; and sham:−0.5±0.5, cb:−0.9±0.5, m1:−0.8±0.3 respectively; F[2,29]<0.3; p>0.7; FIG. 11B). Therefore, no initial performance difference was observed between groups (Pre1) and tDCS did not affect movement execution (Pre2; FIG. 11A). The participant's self-reported ratings of attention, fatigue, and perceived pain were not significantly different across groups (one-way ANOVA: F[2,29]<0.7; p>0.5; FIG. 14). Finally, movement kinematics remained constant between groups and across all six blocks (FIG. 15). Separate ANOVArm comparing group (sham, cb, m1) and block (6) for mean movement duration, reaction time and maximum velocity showed no significant differences for group (F[2,27]<0.9; p>0.3), block F[5,135]<1; p>0.2) or group x block interaction (F[10,135]<0.3; p>0.9; FIG. 15).

During Adapt2, it is evident that the cb group shows less error in epoch one (FIG. 11A). Trial-by-trial analysis of this epoch (ANOVArm: group (sham, cb, m1)×trial (8)) revealed a significant main effect of group (F[2,27]=8; p=0.001), trial (F[7,189]=8; p=0.0005) and group x trial interaction (F(14,189)=2; p=0.01). Separate one-way ANOVAs identified no significant main effect of group at trial one (sham:25±2 degrees, cb:28±2, m1:27±1; F[2,29]=0.7; p=0.5), however by trial four a significant difference was observed (F[2,29]=3; p=0.05). Tukey post-hoc tests revealed the cb group (16±3) showed less error than either the sham (22±2, p=0.03) and m1 (24±2, p=0.006) groups. This indicates that all groups showed a similar amount of error on trial one of Adapt2, however by trial four the cb group showed reductions in error that were not observed in either the sham or m1 groups.

There was a trend towards a significant group effect for both Post1 (sham:−9.4±1.3, cb:−6.2±0.9, m1:−8.3±0.7) and Post2 (sham:−9.3±1.3, cb:−5.9±1.1, m1:−8.5±1.2; F[2,29]>2.2; p<0.1; FIG. 11B). Surprisingly this difference was driven by a reduction of error in the cb group and not an increased amount of error in the m1 group (FIGS. 11A,B), as previously predicted. However, it is important to recall that during deadaptation (Post blocks) at least two factors influence the error in reaching movements: the retention of the previous learned movements and the acquisition of new motor commands resulting from movement errors (Hadipour-Niktarash et al., 2007; Smith et al., 2006).

Initial Vs. End Movement Error

Reaching ‘shooting’ movements are thought to rely on feedforward control with minimal feedback correction (Hadipour-Niktarash et al., 2007; Tseng et al., 2007). In order to quantify whether there was significant changes in angular error from the start to the end of movement, initial angular error was calculated. This was taken as the angular difference between a straight line from the start position to the target, and start position to the positional marker at 80 ms from the onset of each movement (Sainburg and Wang, 2002). For each group, separate ANOVArm compared initial and end angular mean error (time) across block. For all comparisons there was a significant effect for block (F[5,45]>33; p<0.005) however the main effect of time (F[1,9]<1; p>0.7) and time x block interaction (F[5,45]<1; p>0.2) were both not significant. This suggests that there was no significant change in angular direction from the start to the end of reaching movements throughout the experiment.

### Experiment Two: Summary

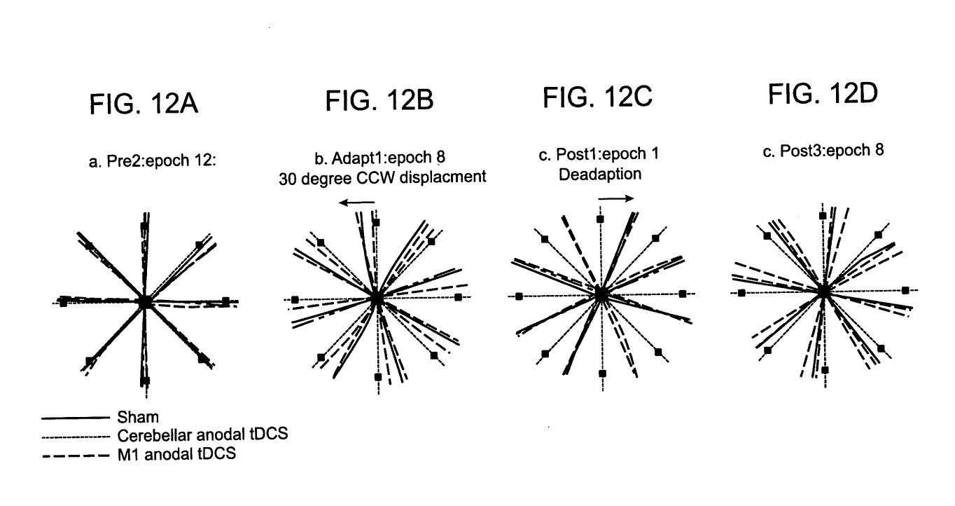

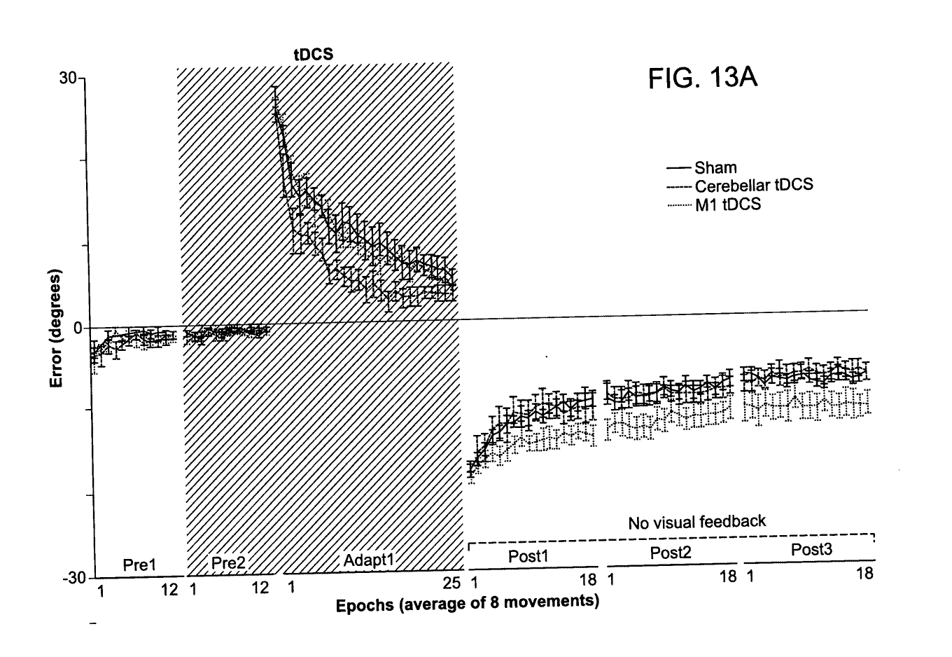

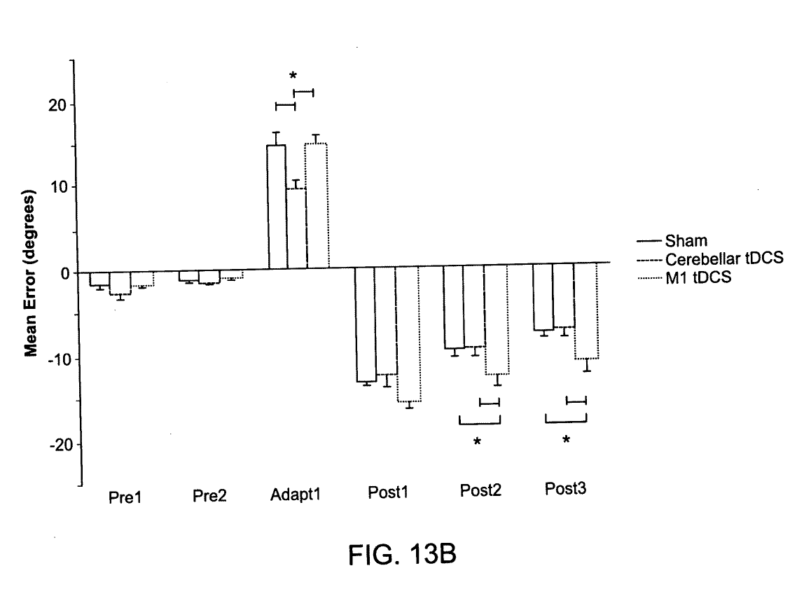

All subjects completed this experiment without complications. The Pre blocks were characterized by accurate performance that was comparable across groups (FIG. 12A). Similarly to experiment one, the cb group showed an increased reduction of error during adaptation (FIG. 12B). At the onset of deadaptation (Post 1), all groups showed the same amount of initial error (FIG. 12C). However, in subsequent trials within Post1 and later in Post2 and Post3 the m1 group remained with marked movement errors indicating increased retention of the recently acquired motor commands (FIG. 12D). These differences between groups are clearly observable when plotting epoch data (FIG. 13A).

Cerebellar tDCS Improves Error Reduction

An ANOVArm, which compared mean error across group (sham, cb, m1) and block (6), showed no significant difference for group (F[2,27]=1.4; p=0.2), however there was a significant main effect of block (F[5,135]=400; p=0.0005) and group x block interaction (F[10,135]=5; p=0.0005). Therefore, separate one-way ANOVAs compared group in each block. Similarly to experiment one, a significant effect of group was found for Adapt1 (F[2,29]=6; p=0.009; FIG. 13B). Tukey post-hoc tests revealed a significant difference between the cb (9±1.3) group and the sham (14.5±1.5; p=0.02) and m1 (14.7±1.1; p=0.02) groups. This again indicates that the cb group experienced a further reduction of error during adaptation (FIG. 13A).

M1 tDCS Increases Retention

When evaluating the deadaptation blocks (Post1,2,3), ANOVArm showed a trend towards a significant group effect for Post1 (sham:−13.2±0.7, cb:−12.6±1.6, m1:−15.7±1; F[2,29]=2; p=0.14; FIG. 13B) and a significant effect for Post2 (sham:−9.5±1, cb:−9.5±1.1, m1:−12.8±1.3; F[2,29]=3; p=0.05) and Post3 (sham:−7.7±0.7, cb:−7.5±0.9, m1:−11.2±1.5; F[2,29]=3.7; p=0.04). For both Post2 and Post3, Tukey post-hoc tests revealed a significant difference between the m1 group and the sham (p<0.05) and cb (p<0.05) groups. Therefore, during deadaptation with no visual feedback, the m1 group showed a persistence of error indicating retention of the recently acquired visuomotor transformation, while the cb group now demonstrated similar performance to sham (FIG. 13A).

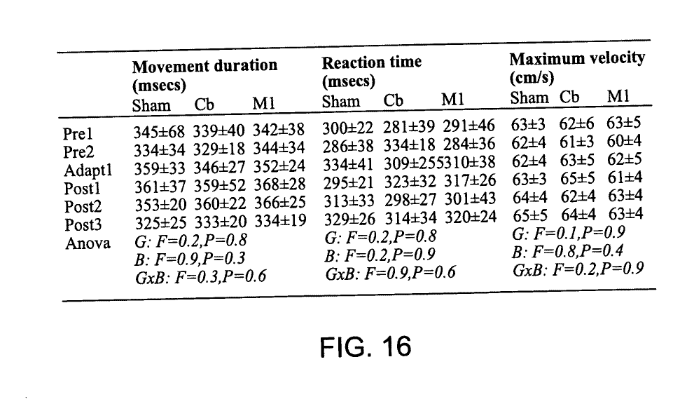

The effects of cerebellar and M1 tDCS could not be explained by differences in baseline performance, in psychological measures or in movement kinematics. In fact, again all groups showed a small CW bias, however there was no significant effect of group for Pre1 (sham:−1.5±0.4 degrees, cb:−2.4±0.8, m1:−1.6±0.4; mean±SE) or Pre2 (sham:−1±0.3, cb:−1.4±0.3, m1:−0.8±0.2) (F[2,29]<1.2; p>0.3; FIG. 13B). Therefore, no initial performance difference was observed between groups (Pre1) and again tDCS did not affect movement execution (Pre2; FIG. 12A). The participant's self-reported ratings of attention, fatigue, and perceived pain were not significantly different across groups (one-way ANOVA: F[2,29]<2; p>0.2; FIG. 14). Finally, movement kinematics remained constant between groups and across all six blocks (FIG. 16). Separate ANOVArm comparing group (sham, cb, m1) and block (6) for mean movement duration, reaction time and maximum velocity showed no significant differences for group (F[2,27]<0.2; p>0.8), block F[5,135]<0.9; p>0.3) or group x block interaction (F[10,135]<0.9; p>0.6; FIG. 16).

### Discussion

This study aimed to dissociate the roles of the cerebellum and M1 during adaptation to a novel visuomotor transformation. During adaptation either anodal or sham tDCS was applied to the cerebellum or M1. Cerebellar tDCS was found to specifically enhance the rate of acquisition without influencing retention. Conversely, M1 tDCS increased retention without affecting acquisition.

When a participant is exposed to a novel visuomotor transformation during reaching they initially make a large error. Over subsequent trials they are able to adapt to the transformation and gradually reduce the error in their movement (acquisition). If the participant is then reintroduced to veridical conditions an error in the opposite direction to the perturbation is observed, with this fading over subsequent trials. This ‘aftereffect’ is thought represent the motor memory of the learnt visuomotor transformation (retention) (Bock et al., 2005; Hadipour-Niktarash et al., 2007).

Previous studies have suggested that the acquisition of a new visuomotor transformation is cerebellar-dependent, and is driven by sensory prediction error. This constitutes the difference between the actual sensory feedback and the expected sensory feedback for a given motor command (Chen et al., 2006; Diedrichsen et al., 2005; Imamizu et al., 2000; Martin et al., 1996; Maschke et al., 2004; Tseng et al., 2007; Weiner et al., 1983).

Evidence that this is cerebellar-dependent mainly originates from patient studies which have shown that cerebellar lesions lead to either an inability or severe impairment in adaptation to visuomotor perturbations imposed by prismatic displacement (Weiner et al., 1983; Martin et al., 1996) or screen-cursor transformations (Rabe et al., 2009). In addition, fMRI studies have associated activation in the ipsilateral cerebellar hemisphere to the active hand with adaptation to a novel visuomotor transformation (Diedrichsen et al., 2005; Flament et al., 1996; Krakauer et al., 2004; Imamizu et al., 2000). Specifically, an inverse relationship was found between the participant's level of performance and the extent of cerebellar activation, supporting the notion that this structure participates in the correction of movement errors (Flament et al., 1996; Imamizu et al., 2000).

In contrast, retention of this newly formed motor memory may be linked to M1 because repetitive-TMS of this area before or after learning can disrupt retention of the adaptation process or more general motor performance (Baraduc et al., 2004; Muellbacher et al., 2002; Richardson et al., 2006). Additionally, disrupting M1 with single-pulse TMS immediately after each trial during adaptation to a novel visuomotor transformation did not influence acquisition but reduced the amount participants subsequently retained (Hadipour-Niktarash et al., 2007).

Although there is a body of evidence suggesting distinct roles for the cerebellum and M1 during adaptive motor learning, this had not been directly tested. Experiments one and two demonstrated that cerebellar tDCS led to a reduced amount of error relative to sham or M1 stimulation in all blocks which involved the rapid correction of movement error. As deadaptation (post) in experiment one was thought to assess retention (Hadipour-Niktarash et al., 2007), it is possible that the observable but non-significant reduction in error for the cerebellar group indicates that cerebellar tDCS led to faster forgetting. However, as previously mentioned during deadaptation, at least two factors influence the amount of error in reaching movements.

With every trial, the participant forgets some of what had previously been acquired, reflecting retention (Smith et al., 2006), and simultaneously learns new motor commands from the movement errors (Hadipour-Niktarash et al., 2007). Therefore to specifically assess retention, experiment two involved deadaptation with no vision. As participants were not exposed to visual movement error the main factor influencing performance was the rate of forgetting. Under these conditions, the cerebellar group now performed similarly to the sham group but M1 tDCS resulted in an increased amount of error, representing enhanced retention. It is believed that these results provide clear evidence that during visuomotor adaptation, tDCS over the cerebellum increases participant's ability to learn from movement error, referred to as acquisition, but has little effect on the subsequent retention of the learnt information. In contrast, M1 tDCS does not influence the ability to learn from movement error however augments the retention of this information.

A recent modeling study attempted to explain the neural network involved during visuomotor adaptation (Tanaka et al., 2009). It is thought that the cerebellum computes a prediction error which is projected to neurons in the cortex. The basic hypothesis of this cerebellar process is that a forward model computes a prediction error between the expected and the observed trajectory of the reaching movement (Miall et al., 1993; Tanaka et al., 2009; Wolpert and Miall, 1996). Gellman et al., (1985) showed that climbing fibers could be reliably activated by cutaneous inputs on the paws of a walking cat, but were inactive if the animal actively used that foot. Thus they suggest that climbing fibers signal unexpected sensory events. This result could reflect the situation in which a sensory prediction fails, in other words sensory inputs are received which are not expected (Wolpert and Miall, 1996).

Within this framework it is believed that the inhibitory output of Purkinje cells is partially modulated by the sensory prediction error signal originating from climbing fiber inputs (Miall et al., 1993; Wolpert and Miall, 1996). As it is thought that cerebellar anodal tDCS increases the output of Purkinje cells (Galea et al., 2009), these cells response to the input of the climbing fibers may have been enhanced. In other words anodal tDCS increased Purkinje cells sensitivity to error. However, although this study was not designed to assess generalization, trial-by-trial analysis of the first epoch during readaptation in experiment one suggests that cerebellar tDCS lead to increased generalization across targets. This was shown as a reduction in error from trial one to trial four which was not observed in the other groups. Therefore, it is proposed that the improvements in error reduction observed with cerebellar tDCS maybe due to two factors: 1) an increase in sensitivity to error and/or 2) broader generalization.

In order to reduce this prediction error, it is thought that the synaptic weights between the neurons in the posterior parietal cortex and motor cortical areas are modified (Tanaka et al., 2009). This leads to a remapping between the reach trajectory in visual space and movement direction in hand space. These changes are reflected by increased activity in neurons of the motor and/or premotor cortex whose preferred direction in hand space matches the required visual trajectory (Tanaka et al., 2009). The effect of M1 tDCS is in accordance with previous reports which dissociate its role in retention from initial acquisition (Baraduc et al., 2004; Hadipour-Niktarash et al., 2007; Muellbacher et al., 2002; Reis et al., 2009; Richardson et al., 2006) and supports Tanaka et al's., (2009) view of the role it may play in the storage of the new visuomotor mappings.

In experiment one, the effect of cerebellar tDCS was present after the cessation of stimulation. This could be a result of either tDCS continuing to modulate cerebellar excitability after the cessation of tDCS (Galea et al., 2009) or due to improved initial adaptation. Although not important to the general conclusions, this issue will be important to understand when using cerebellar tDCS in the context of rehabilitation. For example, does tDCS need to be applied continually during learning or is just one application sufficient to observe long-lasting benefits?

This study provides a clear example of using tDCS as an experimental tool to assess and understand brain function. Importantly, as anodal tDCS augmented normal brain function this study does not suffer from the possible confound of compensation by other neural regions which maybe a problem when testing patient populations or healthy individuals with disruptive TMS protocols. In addition, the results also suggest that tDCS has potential as an intervention to enhance motor learning and retention in both patient and normal populations. The current study implies that an optimal method of using tDCS maybe to stimulate multiple areas which have been shown to have specific and independent functions.

In conclusion, with anodal tDCS one was able to double-dissociate the roles of the cerebellum and M1 during adaptation to a novel visuomotor transformation. Specifically, cerebellar tDCS resulted in faster acquisition with no effect on retention while M1 tDCS led to enhanced retention without influencing acquisition.

Although a preferred embodiment of the invention has been described using specific terms, such description is for illustrative purposes only, and it is to be understood that changes and variations may be made without departing from the spirit or scope of the following claims.

### Incorporation by Reference

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method for increasing learning and effects of training, comprising the steps of:
locating a pair of electrodes on a head of a person so as to be in relation to a specific area of the brain;
applying a desired DC current to the electrodes at a level sufficient to stimulate the tissue of the specific brain area; and
controlling the application of the DC current so as to be applied to the specific brain area at least one of before, during or after such a learning or training event.

2. The method of claim 1, wherein said controlling includes controlling the application of the DC current so as to be applied to the specific brain area during such a learning or training event.

3. The method of claim 1, wherein said controlling includes controlling the application of the DC current so as to be applied to the specific brain area before and during such a learning or training event.

4. The method of claim 1, wherein said controlling includes controlling the application of the DC current so as to be applied to the specific brain area during and after such a learning or training event.

5. The method of claim 1, wherein the electrodes are located at a brain area so that application of the DC current improves a subject ability to acquire one of motor skills or knowledge of the learning or training event.

6. The method of claim 1, wherein the electrodes are located at a brain area so that application of the DC current improves a subject ability to retain one of motor skills or knowledge of the learning or training event.

7. The method of claim 1, wherein the electrodes are applied to the head of a person where the person is one of healthy or a patient after brain lesions.

8. The method of claim 1, wherein the learning or training event includes one of a skilled physical activity or a cognitive task.

9. The method of claim 8, wherein the skilled physical activity is one of typing, playing instruments, sports, dancing, hand skills, rehabilitation activities or fine motor skills for occupations including one of typist, dentists, seamstress, assembly line workers, painters, hand artists like sculpture, video game playing, handling or interfacing with robots, machinist, crane operators, mechanics, drivers, and pilots.

10. The method of claim 8, wherein the cognitive task includes activities that are not solely motoric in nature including one of learning facts, strings or words, language and speech learning, visual coordination and discrimination, activities where perception and sensitivity demands are high, swallowing, and audition.

11. The method of claim 1, wherein said controlling further includes controlling application of the DC current includes applying the DC current such that one electrode delivers anodal stimulation and the other cathodal stimulation.

12. The method of claim 1, wherein said controlling includes selecting of the type of stimulation (anodal or cathodal) and the duration and intensity of the stimulation current so as to be appropriate for the location of stimulation.

13. The method of claim 1, wherein said locating includes locating a plurality of pairs of electrodes, each pair being at a different location and wherein said controlling includes controlling application of the DC current for each pair of electrodes so as to stimulate the specific brain area associated with each pair of electrodes at least one of before, during or after such a learning or training event.

14. A stimulation device for stimulating brain tissue so as to increase learning and effects of training, said stimulation device comprising:
at least one pair of electrodes being configured and arranged so as to maintain the electrodes in relation to a specific area of the brain when the electrodes as disposed on a head of person;
a DC current source being selectively operably coupled to the pair of electrodes; and
a controller operably coupled to the DC current source, the controller being configured and arranged so as to control application of the DC current to the specific brain area; and
wherein the controller controls the current level so as to be sufficient to stimulate the tissue of the specific brain area and the duration of application so that the current is being applied to the specific brain area for at least one of before, during or after such a learning or training event.

15. The stimulation device of claim 14, further comprising a support element to which the electrodes are coupled so that the electrodes are in preselected localization to the head.

16. The stimulation device of claim 15, wherein the support element is adjustable so that the support element and the electrodes is adaptable to different head size while maintaining the desired predetermined electrode position during training or learning.

17. The stimulation device of claim 15, wherein the support element is selected from the group consisting of a cap, hat, headband, or bandana.

18. The stimulation device of claim 15, wherein the support element is configured and arranged so that the electrodes are removably attached to the support element so that the electrodes can selectively attached to an appropriately sized support element.

19. The stimulation device of claim 14, further comprising:
a housing in which the controller and DC current source is disposed;
a plurality of cables for operably coupling the DC current source and the electrodes; and
wherein the housing, controller, current source, cables and electrodes are configured and arranged so that stimulation device is portable and can be worn by the user during learning or training.

20. The stimulation device of claim 14, further comprising a plurality of pairs of electrodes, each pair of electrodes being operably coupled to the controller and DC current source, each pair of electrodes being controllable so as to apply a Dc current to a different brain areas.

21. The stimulation device of claim 20, further comprising a support element to which each pair of electrodes are coupled so the plurality of pairs of electrodes are in preselected localization to different areas of the brain.
